(19) United States
(12) Patent Application Publication
(10) Pub. No.: US 2023/0277167 A1
(43) Pub. Date: Sep. 7, 2023

(54) REAR-VIEWABLE PORT CLOSURE SYSTEM, DEVICE AND METHOD

(71) Applicant: New Wave Endo-Surgical Corp., Coconut Creek, FL (US)

(72) Inventors: Ricardo Alexander Gomez, Lighthouse Point, FL (US); Sandy Heck, Los Angeles, CA (US); Salvatore Castro, Coconut Creek, FL (US)

(73) Assignee: New Wave Endo-Surgical Corp., Coconut Creek, FL (US)

(21) Appl. No.: 18/121,128

(22) Filed: Mar. 14, 2023

Related U.S. Application Data
(60) Provisional application No. 63/317,429, filed on Mar. 7, 2022.

(51) Int. Cl. A61B 17/00; A61B 17/04; A61B 1/313; A61B 17/34; A61B 90/30

(52) U.S. Cl. CPC A61B 17/0057; A61B 1/3132; A61B 17/0469; A61B 17/3423; A61B 90/30

(57) ABSTRACT

A rear-viewable port closure device is provided having a cannular housing, a suture placement assembly, a scope channel defined through the cannular housing for receiving a lens end of a surgical scope, a reflector at a tip region of the of a distal portion of the cannular housing, and an obscured light source, which, when an illumination portion of the light source is activated, provides a reflected rear view to the lens end that is viewable by the surgical scope. The device can be arranged for percutaneous insertion through a defect to an interior cavity of a body, and the reflector can be longitudinally translatable within the interior cavity for adjusting a span of the rear view and a breath of a scope view. A method for rear-viewable, in vivo, suture placement and port closure using the device can include evaluating suture placement via the surgical scope.

### CROSS-REFERENCE TO RELATED APPLICATIONS

This application claims priority to U.S. provisional Pat. application no. 63/317,429 filed on Mar. 7, 2022. In addition, this application is related to U.S. Patent Application No. 15/275,896 filed Sept. 26, 2016 at the U.S. Pat. and Trademark Office (Now U.S. Pat. No. 9,986,987 to Patel et al. entitled “Apparatus and Method for Fascial Closure Device for Laparoscopic Trocar Port Site and Surgery”). Each of the above applications is hereby specifically incorporated by reference in its entirety.

### BACKGROUND

Aspects, features, and principles described herein relate to surgical systems, devices, and related methods for closing a defect, such as an incision or surgical port, and enhanced functionality of the same. More particularly, inventive aspects, features and principles described herein relate to multi-functional surgical port closure devices (a.k.a. suture placement devices) for performing a port closure procedure (a.k.a. suture placement procedure(s)) to close a fascial defect along with the device providing rear view functionality during the procedure, and to corresponding multi-function methods for closing a fascial defect including placing a suture along with enabling rear view (internal) viewability of suture placement actions during the procedure.

The subject matter described in the present application generally relates to laparoscopic surgery and procedures for closing abdominal wall defects. More particularly, the present application is directed to tissue closure devices including surgical suture placement devices, as well as devices that can be used for intra-abdominal suturing, hernia repair, closure of abdominal wall defects, and closure of single port laparoscopy opening.

In the medical field techniques for repairing damaged or diseased tissues are often used. In completing a surgical procedure various surgical methods employing sutures have been used to bind wounds, such as skin, muscles, tendons, internal organs, nerves, and blood vessels. Wound closure devices, such as sutures and staples as well as other repair devices like meshes or patches are used to repair and reinforce wounds. In the case of single port laparoscopy procedures, a single surgical opening can be used for all internal surgical procedures, which can be relatively large compared with multiport procedures. As such, effective closure of the comparatively large single surgical port can have increased complexity that would benefit from internal, rear viewability of suture placement procedures for closing the port.

For discussion purposes and example representations to assist with describing aspects and features for embodiments herein, reference is made to laparoscopic surgical instruments described in U.S. Pat. No. 9,986,987 to Patel et al. (herein “The Patel Patent”). The Patel Patent describes various configurations of an apparatus and related methods for treating tissue openings, such as an endoscopic trocar port opening created and used for a minimally invasive surgical procedure, including a suture placement device configured to rapidly, safely, efficiently and effectively close tissue defects created to access the intra-abdominal cavity during laparoscopic surgical procedures. The device as described can engage adequate tissue adjacent to the defect for enabling robust stitching and port closure that can maintain the pneumoperitoneum during the closure process, which includes techniques involving removal of the trocar from the port opening prior to placement of a fascial suture therein.

Despite the demonstrated effectiveness, reliability and other significant advantages proven by the suture placement device of the Patel Patent, concerns exist regarding the ability to close large and irregular defects. Additional concerns exist regarding their ability to close single port laparoscopic wounds. Current suturing devices are unable to adequately close single port laparoscopic incisions because they require intra-abdominal visualization of the closure process via a laparoscope inserted through an adjacent and separate port but, in single port laparoscopic procedures, there are no other ports available. Closing laparoscopic port wounds without visualization can be dangerous to the patient, as tissues such as bowel or omentum can be inadvertently captured by the suture, potentially leading to injury, reoperation, or death. In addition, continual challenges persist with respect to the duration of surgeries and the likelihood of complications increasing with duration, for which effective time-saving improvements and aids for improving efficiencies are consistently desired and pursued.

Referring now to FIGS. 1A to 1D with respect to prior art devices and related procedures, a diagrammatic plan view of a minimally invasive surgical environment is shown that illustrates significant benefits and advantages enabled through use of an effective configuration of a suture placement device 50′ for port closure procedures. As shown in FIG. 1A, several small diameter ports are typically created through patient tissue for laparoscopic surgical procedures, such as intra-abdominal surgeries. These ports are often formed through the patient’s skin 20′ (i.e., abdominal skin), fat layer 22′, through a fascial layer 24′, and sometimes muscles, which are each kept open as a laparoscopic port via use of a trocar port device 85′. The port depth A′ is determined and a trocar port device 85′ is selected to maintain the port during surgery along with a corresponding suture placement device 50′ for closing the same.

The trocar port device 85′ permits access for surgical instruments, laparoscopic cameras, and the like during surgery while simultaneously sealing the port to prevent the loss of inert gas. Inert gas, such as carbon dioxide, is typically pumped in through one or more ports to create a pneumoperitoneum space below the skin and above the surgical area to provide vital viewability for surgical procedures and maneuver space for surgical instruments and performing procedures. At the conclusion of the surgical procedures, these ports require effective suturing to close the corresponding wound and prevent herniation, which is best performed while maintaining the pneumoperitoneum and starting with effective placement of sutures at the fascial layer to close the port from the inside out.

Suture placement device 50′ greatly enhances the ability of a surgeon to effectively place sutures starting at the fascial layer 24′. The approach of effectively placing fascial sutures first and moving outward has been shown to enhance healing, reduce pain, and greatly reduce the possibility that the port will reopen. An effective method for placing sutures using a suture placement device 50′ is illustrated in FIGS. 1A to 1C, which includes inserting 62′ the distal end of an elongated cannula of the suture placement device 50′ through the corresponding port such that its distal end extends beyond the distal end of the trocar port device 85′, followed by withdrawing 64′ the trocar port device 85′ over the suture placement device 50′ as illustrated in FIG. 1A. The surgeon will select a suture placement device 50′ having a diameter slightly less than the inner diameter of the trocar port device 85′, such that suture placement device 50′ is able to maintain the port opening and prevent significant gas leakage until sutures are placed and the port is closed. The suture placement device 50′ is selected for the port such that its elongate cannula has a longitudinal length B′ sufficient for extending internally beyond the distal end of the trocar port device 85′, spanning the length of the port, and extending proximally an appropriate length for the surgeon to maintain control of the suture placement device and effectively use it for suture procedures.

The suture placement device 50′ is used to position a suture for closing intra-abdominal defects generated by surgical laparoscopic trocar ports and other puncturing devices, and to do so without any exposed sharps, which is enabled due to the suture placement device creating the suture path within the device and the suture being loaded therethrough. This is accomplished, in part, via rotation 66′ of a pivot bar or ‘T-bar’ disposed at the distal end of the suture placement device 50′ about ninety degrees from its longitudinal orientation during insertion, such that the pivot bar is substantially parallel with the fascia layer 24′ and skin 20′ and extends across and beyond the width of the port as shown in FIG. 1B. Thereafter, the suture placement device 50′ is withdrawn 68′ externally until the top portions of the pivot bar are in contact with the fascia layer 24′.

Referring to FIG. 1C, stylet guides disposed on opposite lateral sides of the cannula of the suture placement device 50′ are pushed downward 70′ or extended distally 70′ through the fascia (and muscle as appropriate) until each stylet guide connects with and extends into corresponding openings formed in the rotated pivot bar. The elongate cannula of the suture placement device, the pair of stylet guides, and the pivot bar each define channel segments therein. Upon connection of each stylet guide with and extending into the corresponding openings formed in the rotated pivot bar, the internal channel segments connect to form an uninterrupted internal channel pathway 80 within the suture placement device that extends through the fascia layer along a desired suture path.

As can be seen in FIG. 1D, the internal channel pathway defined through the suture placement device 50′ extends from an entry port 81′ formed at a proximal portion of the device longitudinally downward or distally within a first channel formed in the elongate cannula of the suture placement device to and through a first one of the stylet guides. The channel pathway 80′ continues uninterrupted around and through the rotated pivot bar at the distal end of suture placement device turning into and upwardly or proximally through the second one of the stylet guides. The channel pathway continues proximally through a second channel formed in the elongate cannula to an exit port 83′ formed at the proximal portion of the device. Thus, once suture placement device 50′ has been placed or installed within a port to be closed, and has been prepared for placement of a fascial suture or other suture, the suture placement device 50′ defines therethrough an uninterrupted channel pathway 80′ along a desired suture path.

After the suture thread has been directed through the channel pathway and extends along the pathway, the suture thread is in place to form a highly effective suture through the fascia layer 24′ for closing the defect. Once a suture thread is placed along the channel pathway, thin lateral slots along the stylet guides and pivot bar allow the suture thread to slide out of the channel pathway 80′ and the suture placement device 50′ while maintaining the desired placement through the fascia layer to establish the suture. The stylet guides can be withdrawn upward or proximally and the pivot bar can be rotated back to its initial elongate position to facilitate the suture thread withdrawing from the device while maintaining its suture position, as well as partial or complete proximal withdrawal of the suture placement device 50 out of the port as appropriate for releasing and completing the suture. Thereafter, a suture can be tightened and tied off to close the port at the fascia layer.

It is understood that the same, related or similar surgical devices including other configurations of suture placement devices could also be used for discussion and description purposes with respect to inventive features discussed herein. In addition, advantageous aspects and features of embodiments described herein nonetheless apply to many different and varied surgical devices including various suture placement devices and other surgical devices, and are not limited to use with the example suture placement device. Thus, although aspects and features described herein provide significant benefits and advantages for usage with a suture placement device and/or even the configuration of a suture placement device described as an example for discussion purposes including configurations of the Patel device, it is understood that the subject matter described herein is not so limited.

There is a need in the field for a Laparoscopic device capable of overcoming various drawbacks and challenges of conventional devices and significantly improving techniques, devices and related technology for closing defects. Further, there is a need for improved methods and techniques for enhanced defect closure procedures for single port laparoscopic procedures along with multi-port laparoscopic procedures, closure of irregular shaped defects or wounds and for mitigating hernia concerns, as well as for laparoscopic devices for supporting the same.

### SUMMARY

One general aspect discussed herein along with example arrangements and methods includes the incorporation of a scope channel into a port closure device, with the scope channel operable for permitting the passage of a surgical scope from a proximal end of the port closure device towards its distal end. This can provide advantages for visualizing closure of a defect from the inside of the abdomen or other portion of the body without requiring a separate port for the surgical scope. These and related advantages can be particularly useful is single port cases, in which only one access port is available. Because these single port cases often utilize large (20-30 mm) ports, they typically require closure with sutures and that often necessitate the use of a port closure device.

According to aspects and features of example configurations described herein, a rear-viewable port closure device is provided that can include a cannular housing operable for percutaneous insertion through a defect to an interior cavity of a body that has a proximal portion, an opposite distal portion, and a longitudinal axis oriented therebetween, a suture placement assembly connected to the cannular housing at the distal portion, a scope channel defined through the cannular housing that is operable for receiving a lens end of a surgical scope and retaining the lens end at a view position along the longitudinal axis, and a reflector at a tip region of the distal portion having a proximal reflective surface and an opposite distal surface, in which the proximal reflective surface is oriented substantially perpendicular to the longitudinal axis and is operable to reflect a rear view of the suture placement assembly to the lens end. The device further includes an obscured light source connected to the cannular housing having an illumination portion disposed outside of the rear view and outside of a viewable span of the lens end, which is operable for illuminating the rear view and the viewable span when the illumination portion is activated, such that when the illumination portion is activated, the rear view and the viewable span are illuminated and viewable by the surgical scope without the illumination portion being viewable through the surgical scope.

Implementations can include one or more of the following features. The rear-viewable port closure device reflector can be movable with respect to the cannular housing between a first position and at least one second position, in which the at least one second position corresponds with a deployed state of the suture placement assembly and the reflector is operable to reflect the rear view to the lens end of the surgical scope when in the at least one second position. The reflector can be translatable parallel to the longitudinal axis with respect to the cannular housing between the first position and the at least one second position, in which the first position can correspond with an insertion-removal state of the suture placement assembly operable for insertion through the defect to the interior cavity and for removal from the interior cavity through the defect. The plurality of second positions can include a preliminary gap position in which the reflector is distally translated a gap distance apart from the first position for enabling deployment of the suture assembly, a distal extension position in which the reflector is distally translated a first distance from the preliminary gap position for providing the rear view to the lens end of the surgical scope, and a proximal extension position in which the reflector is proximally translated a second distance from the preliminary gap position for providing the rear view to the lens end of the surgical scope. In addition, the distal extension position and the proximal extension position can fall within a reflector adjustment range for enabling user adjustment of at least one of the rear view and the viewable span.

In some implementations, the reflector can include a concave reflector for which the proximal reflective surface is concave, such that the proximal reflective surface is operable to provide the rear view to the lens end as a magnified view having a magnification factor greater than one (1.0) and the magnification factor and a breadth of the rear view are adjustable based on the at least one second position. In some implementations, the reflector can include a convex reflector for which the proximal reflective surface is convex, such that the proximal reflective surface is operable to provide the rear view to the lens end as a diminished view having a magnification factor less than one (1.0) and the magnification factor and a breadth of the rear view are adjustable based on the at least one second position. In some implementations, the reflector can include a mirror with a transparent sheet having a transparent proximal surface and a distal opaque surface, in which a proximal face of the distal opaque surface is operable to reflect light and an opposite distal face of the distal opaque surface is operable to block light. The mirror can include one of a concave mirror, a convex mirror, and a substantially flat mirror, such that the substantially flat mirror is operable to provide the rear view to the lens end as a real image substantially unmagnified and undiminished and a breadth of the rear view is adjustable based on the at least one second position.

In some implementations, the illumination portion can be translatable parallel to the longitudinal axis with respect to the cannular housing along with translation of the reflector. The illumination portion can be operable for indirectly illuminating the rear view and portions of the interior cavity within the viewable span. In some implementations, the illumination portion can be located at at least one of the following locations: distally behind the distal surface of the reflector; and at an outboard side of at least one of the reflector support arms. In some implementations, the obscured light source can include a light pipe extending through one of the plurality of support arm channels and forming one of the plurality of support arms, in which a region of the light pipe forms the illumination portion. The light pipe can have an opaque exterior substantially covering an outer surface of the light pipe without covering the outer surface at the illumination portion, such as a sleeve covering or an opaque coating. A proximal end of the light pipe can include an adaptor operable for attaching to an illumination source, which can include an attachment to the surgical scope or an independent attachment to light source or power supply.

In some implementations, an exterior region of the cannular housing extending along the longitudinal axis defines an elongate housing envelope and the suture placement assembly is movable between an insertion-removal arrangement and a suture-placement arrangement, in which a perimeter of the suture placement assembly is substantially the same as or fits within the elongate housing envelope when in the insertion-removal arrangement. In addition, portions of the suture placement assembly can extend laterally away from the longitudinal axis and outside of the elongate housing envelope when in the suture-placement arrangement corresponding with a deployed state of the rear-viewable port closure device for establishing at least one suture pathway that includes a transverse suture pathway region across the defect. The rear view can be operable to include the transverse suture pathway region of the at least one suture pathway.

In some implementations, the rear-viewable port closure device is operable for selective placement between the deployed state and a non-deployed state for insertion and removal of the rear-viewable port closure device through the defect to the interior cavity, in which the non-deployed state can include the suture placement assembly in the insertion-removal arrangement and the reflector is located at the first position located immediately adjacent to a distal end of the suture placement assembly. The rear view can be operable to include a transverse suture pathway region and suture bites for one or more suture pathways when the rear-viewable port closure device is disposed through the defect and the suture placement assembly is in a suture-placement arrangement operable for establishing at least one suture pathway having a transverse suture pathway region extending across the defect for implementing suture bites centered about a perimeter of the defect. In some implementations, when the obscured light source is activated light can travel from the obscured light source to the cone assembly such that the cone assembly emanates light.

According to aspects and features described herein along with example configurations, a method for rear-viewable, in vivo, suture placement and port closure using a rear-viewable port closure device described above can include inserting the cannular housing through the defect to the interior cavity, deploying and placing the suture placement assembly in a suture-placement arrangement for establishing one or more suture pathways each having a transverse suture pathway region extending across the defect and implementing suture bites centered about a perimeter of the defect, and activating the obscured light source. The method can further include evaluating a suture placement configuration based on the suture-placement arrangement, which can include viewing the rear view via a proximal view end from the surgical scope, placing a suture through each of the one or more suture pathways including through the transverse suture pathway region across the defect, placing the suture placement assembly in an insertion-removal arrangement, and removing the rear-viewable port closure device from the defect.

In some implementations, the reflector can be operable for selective translation parallel to the longitudinal axis between a first position located adjacent to a distal end of the suture placement assembly when the suture placement assembly is in the insertion-removal arrangement and at least one second position distally translated away from the first position, and the method can further include translating the reflector from the first position to the at least one second position substantially simultaneously with the action of deploying and placing the suture placement assembly in the suture-placement arrangement. Further, the method can include translating the reflector from the at least one second position to the first position substantially simultaneously with the action of placing the suture placement assembly in the insertion-removal arrangement. The at least one second position can include a plurality of second positions including a preliminary gap position and at least one of a distal extension position or a proximal extension position.

In some implementations, the method can include translating the reflector from the first position to a preliminary gap position where the reflector is distally translated a gap distance apart from the first position for enabling deployment of the suture assembly, which can be performed substantially simultaneously with the action of deploying and placing the suture placement assembly in the suture-placement arrangement. Further, substantially simultaneously with the action of evaluating the suture placement configuration, the method can further include translating the reflector from the preliminary gap position to one of the distal extension position and the proximal extension position, in which translating the reflector from the preliminary gap position to the distal extension position includes distally translating the reflector a first distance from the preliminary gap position to the distal extension position. Translating the reflector from the preliminary gap position to the proximal extension position can include proximally translating the reflector a second distance from the preliminary gap position to the proximal extension position. The distal extension position and the proximal extension position can fall within a reflector adjustment range for enabling user adjustment of at least one of the rear view and the viewable span. Translating the reflector from the preliminary gap position to one of the distal extension position and the proximal extension position can enable user adjustment of at least one of the rear view and the viewable span.

Other aspects and advantages according to aspect, features, innovative concepts and implementations discussed along with example devices, arrangements and methods will become apparent from the following detailed description, which when taken in conjunction with the accompanying drawings illustrates by way of example innovative aspects, features and concepts described herein.

### BRIEF DESCRIPTION OF THE DRAWINGS

FIG. 1A is a diagrammatic plan view of a minimally invasive surgical environment operable to use an example prior art surgical device in the form of a suture placement device that can be supported by aspects and features of guide wire advancers discussed herein, for which the suture placement device and corresponding diagrammatic surgical environment are described and shown in FIGS. 1A to 1D are for discussion purposes to assist with describing aspects and features described herein; To Wit, the diagrammatic surgical environment shown includes a plan view of a prior art trocar device installed through a laparoscopic surgery port formed through patient tissue along with a front plan view of an example prior art suture placement device inserted therethrough in preparation for port closure procedures.

FIGS. 1B and 1C are front plan views of the prior art suture placement device of FIG. 1A and depicting example actions pertaining to arranging the suture placement device of FIG. 1A for placement of a suture, for which aspects and features discussed herein are related.

FIG. 1D is a front plan view of the prior art suture placement device of FIG. 1A depicting introduction of a guide wire along a channel pathway established through the device in preparation for placing the suture.

FIG. 2A is a front perspective view of a schematic representation of an example rear-viewable port closure device according to aspects and features described herein.

FIG. 2B is a detail view of a tip region of a distal portion of the rear-viewable port closure device of FIG. 2A.

FIG. 2C is a right, front perspective view of a schematic representation of another example of a rear-viewable port closure device according to aspects and features described herein that is substantially the same as the example rear-viewable port closure device of FIG. 2A except for having a different example obscured light source arrangement as described below, which is shown with outer portions of the cannular housing removed.

FIG. 2D is a front plan view of a further schematic representation of an example rear-viewable port closure device according to aspects and features described herein that is substantially the same as the example rear-viewable port closure devices of FIG. 2A and FIG. 2C except for having another example obscured light source arrangement as described below, which is shown during use as part of a port closure surgical procedure having the cannular housing inserted through a defect via trocar to an interior cavity similar to the procedure described along with FIG. 1A.

FIG. 2E is a front plan view of the rear-viewable port closure device of FIG. 2D shown with the trocar removed or as part of a port closure surgical procedure having the cannular housing inserted through the defect without a trocar extending through the defect, in which the rear-viewable port closure device is positioned for placing a suture and optionally injecting a subdermal anesthetic.

FIG. 2F is a front plan view of the rear-viewable port closure device of FIG. 2D shown positioned for placing the suture and with a reflector positioned for providing a rear view of the suture placement to a lens end of a surgical scope.

FIG. 2G is a detail view of the tip region of the distal portion of the cannular housing for the rear-viewable port closure device of FIG. 2D, in which the detail region is indicated in FIG. 2F.

FIG. 2H is an example depicting a top view of the reflector and cone assembly of the rear-viewable port closure device of FIG. 2D, which can be seen through the surgical scope including the reflected rear view.

FIG. 3 is a schematic depiction of an example Method 400 for using a rear-viewable port closure device according to aspect and features described herein.

FIG. 4A is front, plan, cross-sectional view of a schematic depiction of an additional example rear-viewable port closure device according to aspects and features described herein, which includes a longitudinally translatable reflector and is depicted in an insertion-removal arrangement.

FIG. 4B is a front plan cross-sectional view of the rear-viewable port closure device of FIG. 4A depicted in a deployed arrangement with the reflector distally translated.

FIG. 5 is a front left perspective view of a further example rear-viewable port closure device according to aspects and features described herein, which is depicted in a compact, insertion-removal arrangement.

FIG. 6 is a front right perspective view of the rear-viewable port closure device of FIG. 5 depicted in the compact, insertion-removal arrangement.

FIG. 7 is a detail view of a distal portion of the cannular housing of the rear-viewable port closure device of FIG. 5, which is indicated in FIG. 6 at the tip region thereof.

FIG. 8 is a right plan view of the rear-viewable port closure device of FIG. 5 depicted in the compact, insertion-removal arrangement.

FIG. 9 is a left plan view thereof.

FIG. 10 is a front plan view thereof.

FIG. 11 is a rear plan view thereof.

FIG. 12 a top view thereof.

FIG. 13 is a top view thereof shown without the surgical scope inserted through a scope channel formed through the cannular housing.

FIG. 14 is a bottom view thereof.

FIG. 15 is a bottom view thereof shown without the dome assembly.

FIG. 16 is a front right exploded perspective view thereof.

FIG. 17A is a partial exploded perspective view thereof showing components of the obscured light source, the dome assembly and the reflector.

FIG. 17B is a partial exploded perspective view thereof showing cover portions of the cannular housing that form portions of the scope channel and channels for the reflector support arms.

FIG. 18 is a front right perspective view of the rear-viewable port closure device of FIG. 5, which is depicted in a deployed arrangement.

FIG. 19 is a right plan view thereof depicted with the surgical scope inserted through the scope channel and with a distal, right cover portion of the cannular housing removed for viewing the deployed suture placement assembly.

FIG. 20 is a left plan view thereof.

FIG. 21 is a front plan view thereof.

FIG. 22 is a rear plan view thereof.

FIG. 23 a top view thereof.

FIG. 24 is a top view thereof.

FIG. 25 is a bottom view thereof.

FIG. 26 is a top cross-sectional view thereof as taken from line 26-26 shown in FIG. 22.

FIG. 27A is an exploded perspective view thereof.

FIG. 28 is a front plan view of a distal portion thereof and of the cannular housing depicted in a first position corresponding with the compact insertion-removal arrangement.

FIGS. 29, 30 and 32 are front plan views thereof depicted with the reflector distally extended to a preliminary position corresponding with a deployed arrangement.

FIGS. 31 and 34 are front plan views thereof depicted with the reflector proximally translated from the preliminary position to a proximal extension position.

FIGS. 33 and 35 are front plan views thereof depicted with the reflector distally translated from the preliminary position (FIG. 33) or from the proximal extension position (FIG. 35) to a distal extension position.

FIG. 36 is a schematic depiction of an example Method 600 for using a rear-viewable port closure device, such as the example rear-viewable port closure device of FIG. 5, which is described as part of Method 400 shown in FIG. 3 according to aspect and features described herein.

FIG. 37 is a front plan view of a schematic depiction of distal portions of an additional rear-viewable port closure device according to aspects and features described herein, which includes a convex reflector.

FIG. 38 is a front plan view of a schematic depiction of distal portions of another rear-viewable port closure device according to aspects and features described herein, which includes a flat reflector that can include a flat mirror.

FIG. 39 is a front plan view of a schematic depiction of a flat mirror that can be used as the flat reflector with the rear-viewable port closure device of FIG. 38.

### DETAILED DESCRIPTION

For the purposes of promoting an understanding of various inventive aspects, features and principles described herein, reference will now be made to the example configurations and arrangements illustrated in the drawings along with language describing the same. It will nevertheless be understood that no limitations regarding scope is thereby intended. Any alterations and further modifications of the inventive features shown or described herein, and any additional applications of inventive principles shown or described herein, which would occur to one skilled in the relevant art and having possession of this disclosure, are to be considered within the scope of the invention.

Aspects, features and concepts described herein pertaining to surgical apparatus, methods, and devices are directed to apparatus and methods for closing tissue openings including puncture wounds, hernias, endoscopic trocar port openings used in minimally invasive surgical procedures, other abdominal wall defects in the fascia, and single port laparoscopy openings. Aspects, concepts and features pertaining to a suture placement device are discussed for rapidly, safely, efficiently and effectively repairing minimally invasive surgical opening, hernias and other tissue defects in a human body including options for rear viewing of suture placement actions at a distal end of the device from an internal perspective.

Example devices as described herein can be able to obtain adequate tissue adjacent to the tissue defect to provide a strong closure, to maintain pneumoperitoneum needed for appropriate visualization of the peritoneal contents during the closure process, and to protect the vital structures within the abdominal cavity in the vicinity and the healthcare provider for risk of injury.

There is currently no single device that provides means similar to examples discussed herein for suturing an internal organ defect and performing fascial port closure with simultaneous multiple tissue bites, or for enabling rear viewing of surgical actions at a distal end of the device from an internal perspective. These and other novel and innovative concepts and improvements discussed herein can be performed using a single device according to aspects and features described herein along with example arrangements, which can include for example applying a nerve block with the same device and method for performing fascial port closure. Other enhancements, concepts and features described herein can include implementation of significantly improved closures, which can be further improved by adhering to JOHNSSON-ISRELSON rules and guidelines for fascial closure, such as implementing symmetrical and evenly spaced suture bites equidistant from a center of a defect, such as about 1 cm.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used herein, the term “target workspace” refers to anything within or pertaining to the endoscopic work cavity including the body of the patient, tissues and organs within the cavity, and tissue defining the cavity, and also to support structures for the MIS procedure including a cover and cannula supports, instruments and related attachments or medical implements including needles, suture materials, implants, meshes, etc. As used herein, the term “target tissue” refers to any tissue or organ that interacts with the target workspace including tissues and organs of the patient, natural tissues and organs introduced to the target workspace including natural transplant tissues and organs, artificial tissues and organs including mechanical or electro-mechanical organs, and tissue and organ assist devices such as pacemakers, mesh material, artificial skin and the like.

As used herein, a surgical device or tool or clinical instrument refers to a medical instrument having contact surfaces that are operable to engage organs, tissues and/or portions of a surgical cavity or wound to thereby move, hold, lift, retain, suture or otherwise engage, interface or make contact with the target tissue and perform clinical functions as appropriate for the surgical environment. The term “flexible” in association with a part, such as a mechanical structure, component, or component assembly, should be broadly construed. In essence, the term means the part can be repeatedly bent and restored to an original shape without harm to the part. Certain flexible components can also be resilient. For example, a component (e.g., a flexure) is said to be resilient if possesses the ability to absorb energy when it is deformed elastically, and then release the stored energy upon unloading (i.e., returning to its original state). Many “rigid” objects have a slight inherent resilient “bendiness” due to material properties, although such objects are not considered “flexible” as the term is used herein.

A flexible part can have infinite degrees of freedom (DOF’s). Flexibility is an extensive property of the object being described, and thus is dependent upon the material from which the object is formed as well as certain physical characteristics of the object (e.g., cross-sectional shape, length, boundary conditions, etc.). For example, the flexibility of an object can be increased or decreased by selectively including in the object a material having a desired modulus of elasticity, flexural modulus, and/or hardness. The modulus of elasticity is an intensive property of (i.e., is intrinsic to) the constituent material and describes an object’s tendency to elastically (i.e., non-permanently) deform in response to an applied force. A material having a high modulus of elasticity will not deflect as much as a material having a low modulus of elasticity in the presence of an equally applied stress. Thus, the flexibility of the object can be decreased, for example, by introducing into the object and/or constructing the object of a material having a relatively high modulus of elasticity.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to an actuation shaft) would be the proximal end of the tool.

Further, specific words chosen to describe one or more embodiments and optional elements or features are not intended to limit the invention. For example, spatially relative terms-such as “beneath”, “below”, “lower”, “above”, “upper”, “proximal”, “distal”, and the like-can be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures were turned over, elements described as “below” or “beneath” other elements or features would then be “above” or “over” the other elements or features. Thus, the term “below” can encompass both positions and orientations of above and below. A device can be otherwise oriented (e.g., rotated 90 degrees or at other orientations) and the spatially relative descriptors used herein interpreted accordingly. Likewise, descriptions of movement along (translation) and around (rotation) various axes include various spatial device positions and orientations. The combination of a body’s position and orientation define the body’s pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, instrument, and variants thereof, can be interchangeably used.

Example device configurations are generally shown having an internal scope channel, an obscured light source, and reflector, which can be incorporated into various port closure device configurations. Example configurations and arrangements are shown, for instance, incorporated into configurations based on devices known as a CARTER-THOMASON device and an M-CLOSE device as illustrative examples, but other devices that would benefit from these improvements including configurations based on devices known as a WECK EFX device, a SHIELD device, a LAPROSHARK device, a NEOCLOSE device, a VERSO ONE device, a CROSSBOW device, a NEATSTITCH device, and other similar devices.

The reflector can be operable with a fixed extension length or a variable extension length to accommodate various surgical procedures and corresponding needs for a breadth or size of the scope view and/or a span of the reflected rear view, which can be associated with defect sizes and port closure device configurations. Movement or extension of the reflector can be controlled with a slidable member, and/or it can be extended as the surgical scope traverses the scope channel. Further, a position for the lens end of the surgical scope within the scope channel can include a fixed position or can include a range of positions at a distal end region of the scope channel.

The reflector can include a mirror or other reflector device having a reflective proximal surface that would allow an image of the interior abdominal wall to be reflected to a lens end of a surgical scope as a rear view. As an alternative to configurations having a substantially opaque reflector, the reflective proximal surface can be translucent or semi-transparent for providing a forward view along with the rea view. A breadth or range of the scope view can be larger than a span of the rear view for viewing distal (‘forward’) portions of an interior cavity beyond a perimeter region of the reflector.

The proximal reflective surface can be of a variety of shapes and curvatures, including flat, concave or convex, depending on the viewing requirements for the given port closure device.

The lighting necessary to see the interior abdominal wall can be provided as an obscured light source that is substantially outside the view of the scope for avoiding whitewash or light noise conditions that can interfere with or diminish viewability of the interior cavity through the scope, which can occur based on directly viewing a light source through the surgical scope. The obscured light source can include an illumination portion coming from the scope itself, such as a laterally directed illumination portion (e.g., light-emitting diode or LED) disposed proximally with respect to the scope lens such that the light can be directed to the interior cavity or other target. Further, light from the obscured light source can come from the port closure device itself, such as one or more LEDs and/or light pipes. An example configuration described herein can include a dedicated light source connector located, for example, at a proximal portion of the port closure device for ease of access, which can be connected to one or more illumination portions located at a distal portion of the device via a connector tube or channel, light pipe or other intermediary. The one or more illumination portions can include, for example, an exposed portion of a light pipe, an LED, and/or lenses, prisms or fiberoptic channels. The light can be directed into a clear nose cone, extension arm or other device components that can glow for providing necessary light within the interior cavity.

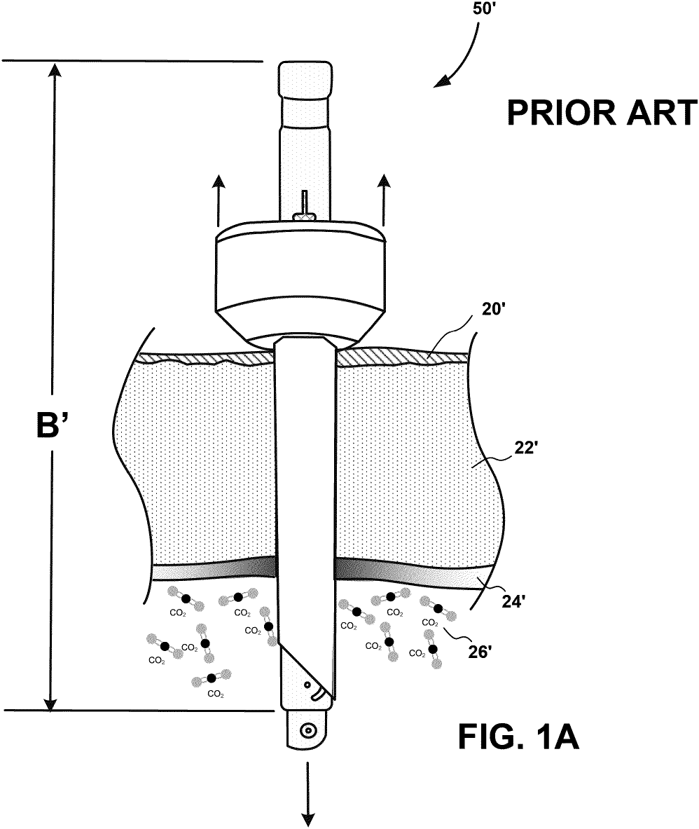

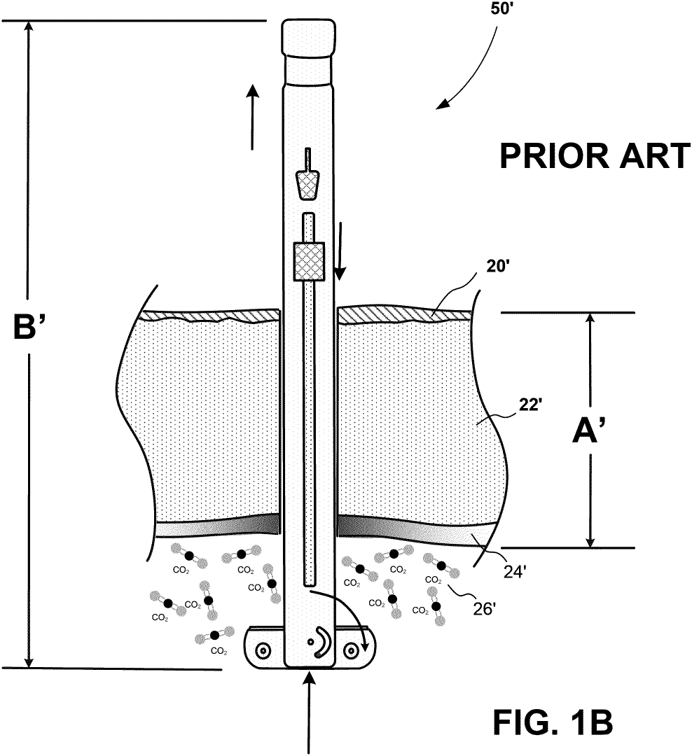

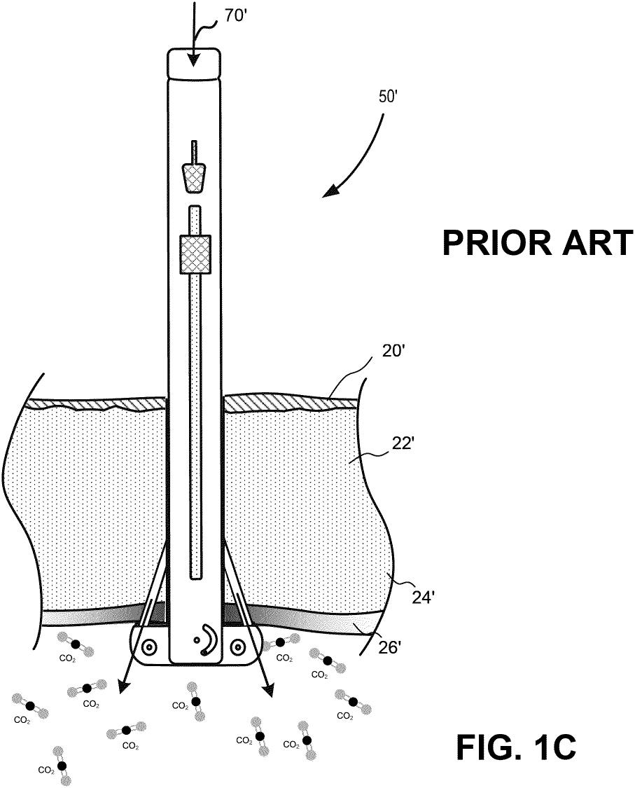

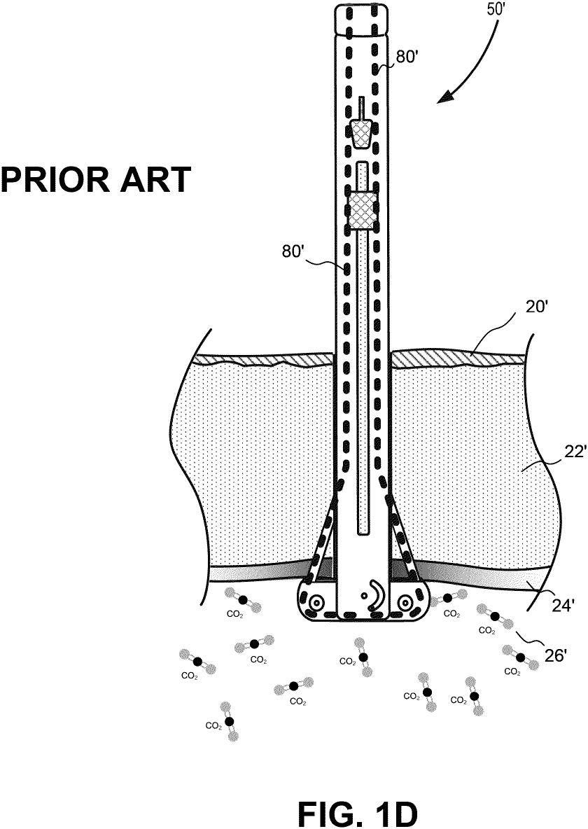

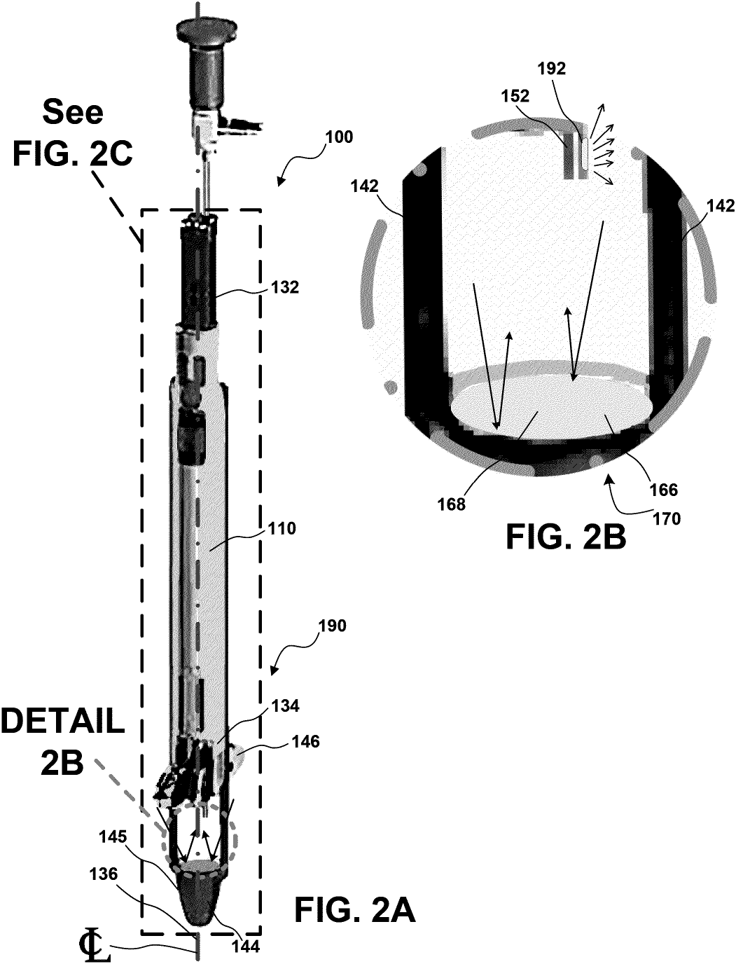

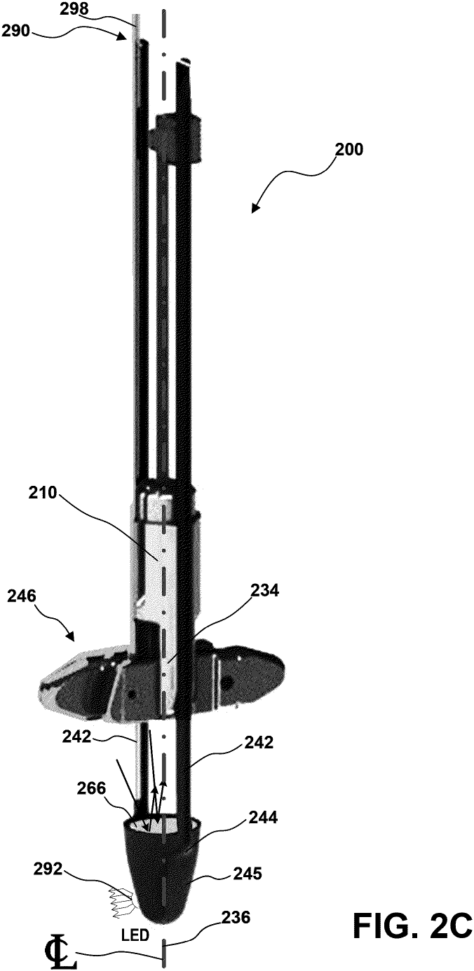

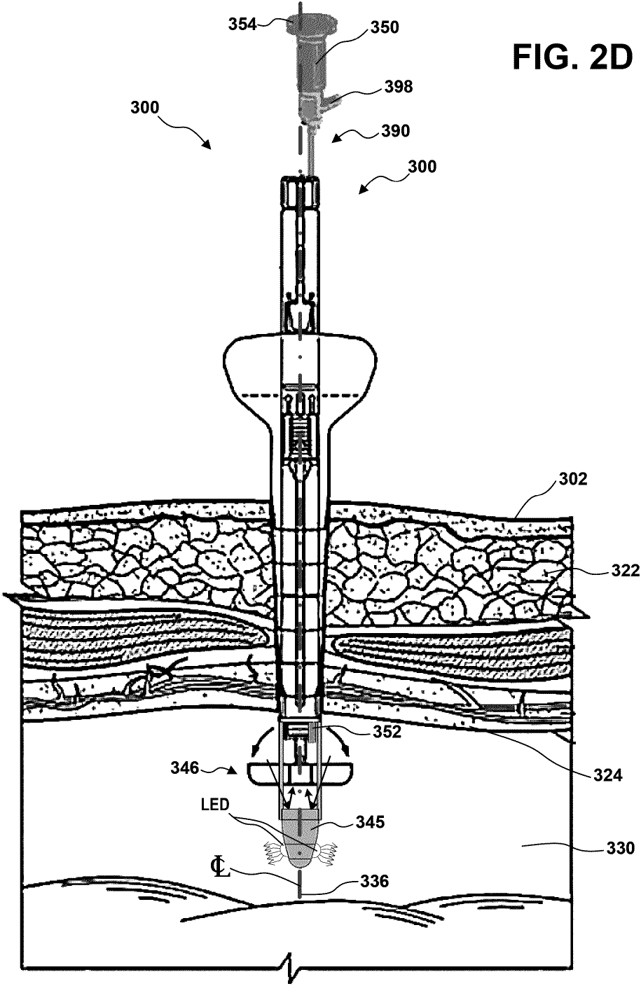

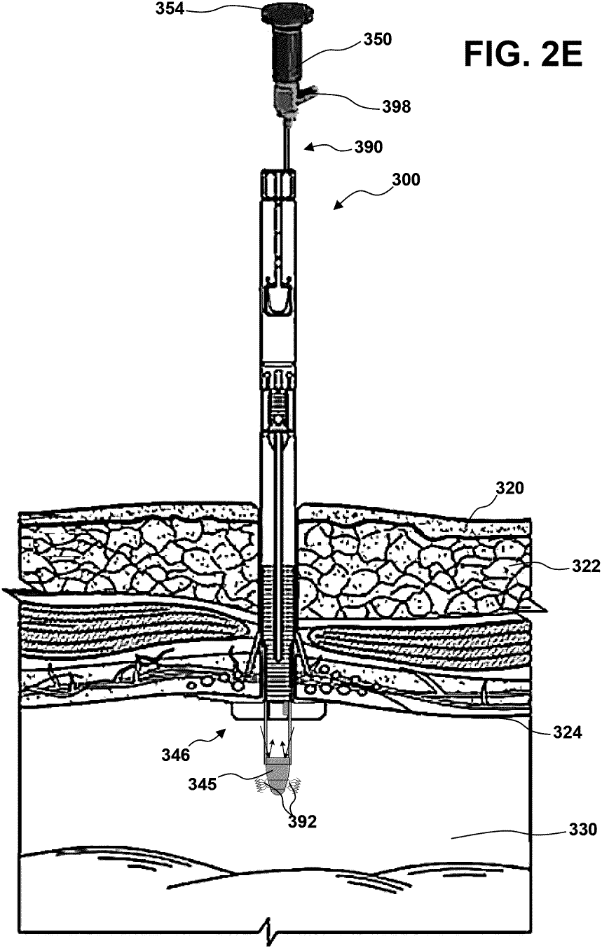

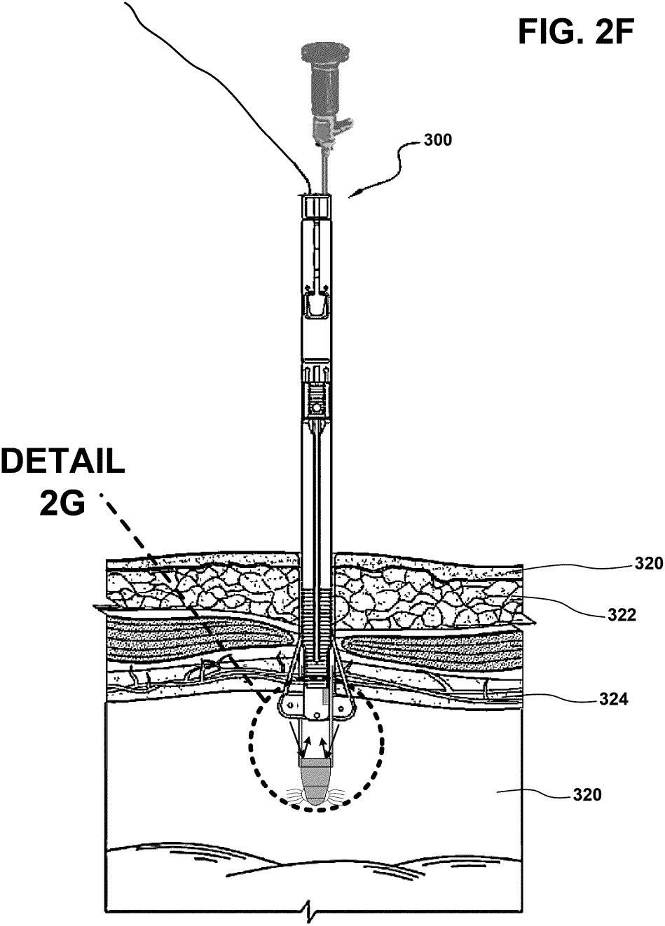

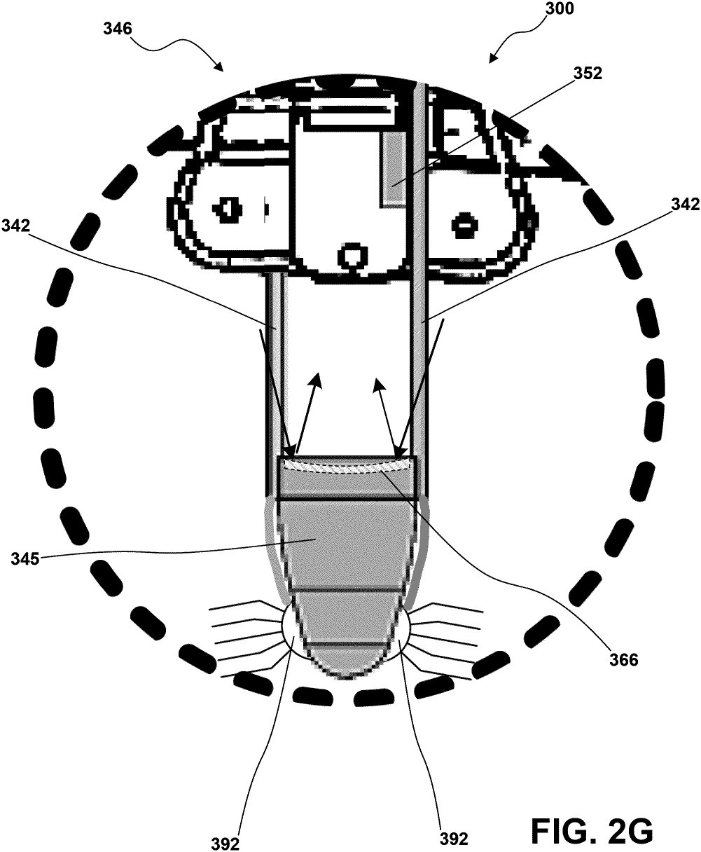

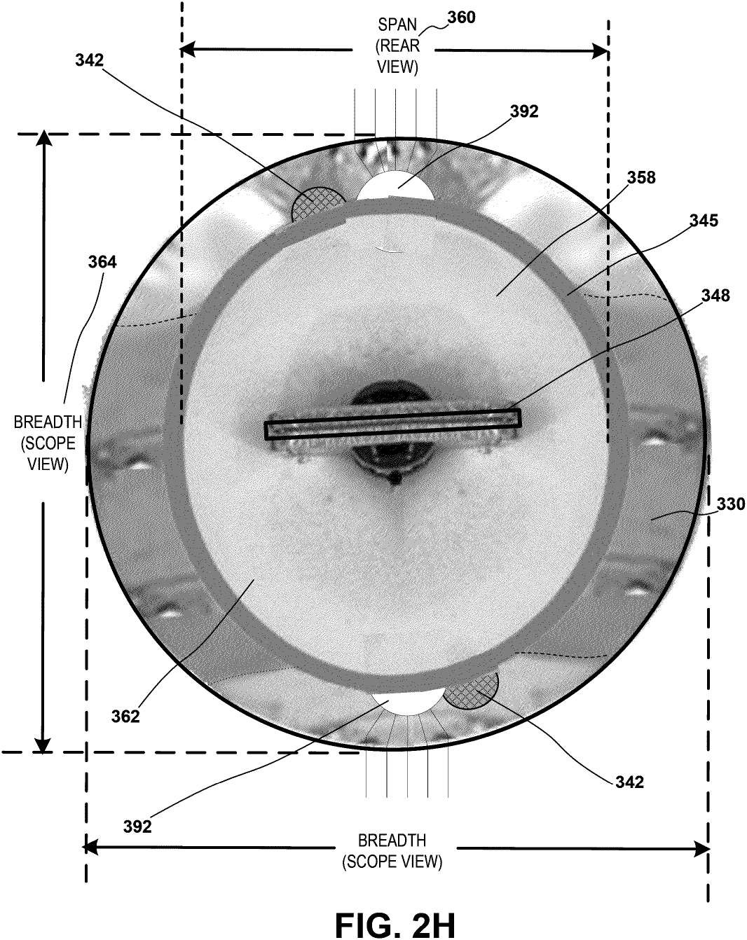

Referring now to FIGS. 2A and 2B, a schematic representation of an example rear-viewable port closure device 100 is generally shown, which is depicted in FIG. 2A in a deployed arrangement. The rear-viewable port closure device 100 can be operable for percutaneous insertion through a defect to an interior cavity of a body like the example prior art descriptions above for the prior art devices and methods of FIGS. 1A to 1D, and generally includes the aspects and features except as described herein. As such, like numbers refer to like features.

The rear-viewable port closure device 100 can include a cannular housing 110, a suture placement assembly 146, a scope channel (not shown) (see e.g., FIG. 2H) defined through the cannular housing, a reflector 166, and an obscured light source 190 connected to the cannular housing. The cannular housing 110 can include a proximal portion 132, an opposite distal portion 134, and a longitudinal axis 136 oriented therebetween.

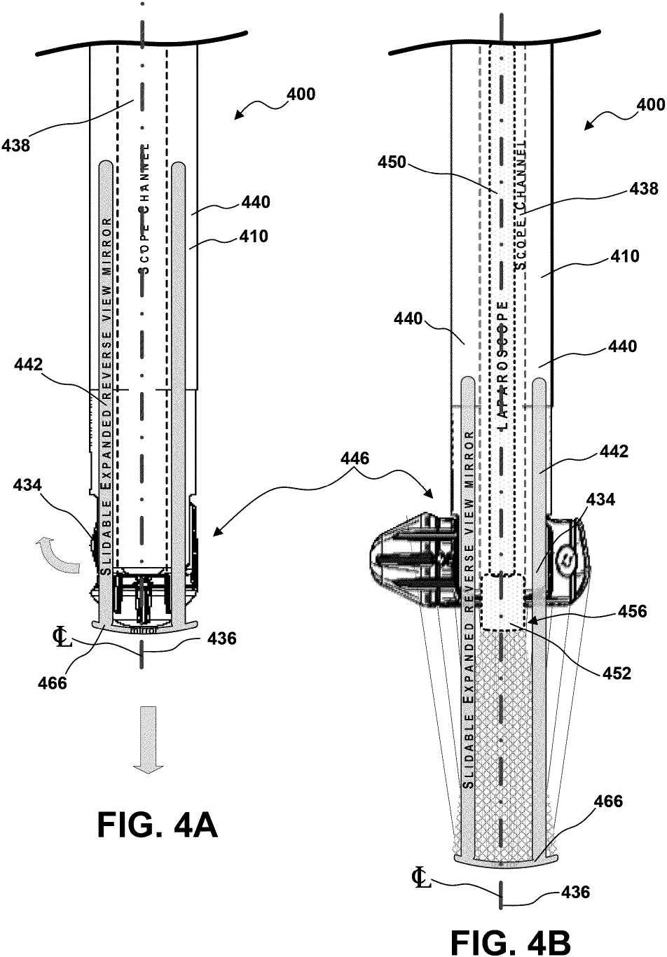

The scope channel (not shown) (see e.g., FIGS. 4A and 4B) can be defined through the cannular housing 110 and operable for receiving a lens end 152 of a surgical scope 150 and retaining the lens end at a view position 156 along the longitudinal axis 136. The suture placement assembly 146 can be connected to the cannular housing 110 at the distal portion 134, and the reflector 166 can be located at a tip region 144 of the distal portion. The reflector 166 can have a proximal reflective surface 168 and an opposite distal surface 170, in which the proximal reflective surface can be oriented substantially perpendicular to the longitudinal axis. The proximal reflective surface 168 can be operable to reflect a rear view of the suture placement assembly 146 to the lens end 152. The obscured light source 190 can be connected to the cannular housing 110 and can include an illumination portion 192 disposed outside of the rear view and outside of a viewable span (not shown) (see e.g., FIG. 2H) of the lens end 152. The illumination portion 192 can be operable for illuminating the rear view and the viewable span when the illumination portion is activated for providing illumination within the interior cavity, such that, when the illumination portion is activated, the rear view and the viewable span are illuminated and viewable by the surgical scope 150 without the illumination portion viewable through the surgical scope.

As best seen in FIG. 2B, the obscured light source 190 can be integrated within the surgical scope, such as from a light-emitting diode (LED) or other light source from the surgical scope, but in an obscured arrangement such that light emitted from illumination portion 192 is not directly viewable by the surgical including by the rear view reflected to the lens end 152. For instance, the illumination portion 192 for the example shown in FIG. 2B emits light laterally from a side portion of a distal end portion of the surgical scope.

Referring now to FIG. 2C, a schematic representation of another example rear-viewable port closure device 200 is shown that generally includes the same aspects and features as rear-viewable port closure device 100 except as described herein. As such, like numbers refer to like features. The rear-viewable port closure device 200 is shown without exterior cover portions of the cannular housing 210 shown for exposing an arrangement of interior components. Further, the rear-viewable port closure device 200 is depicted without a surgical scope inserted through the cannular housing 210.

The rear-viewable port closure device 200 primarily differs from rear-viewable port closure device 100 in that it the obscured light source 290 includes an illumination portion 292 located in the tip region 244 of the cannular housing distal portion 234. The illumination portion 292 can be disposed at a lower portion of the cone assembly 245, which can be substantially opaque for blocking direct view of the illumination portion 292 by a lens end of the surgical scope (not shown) when inserted therein. As such, the illumination portion 292 can illuminate the interior cavity during usage for a surgical procedure pertaining to placing one or more suture for closing a defect via indirect light being projected to the lens end of the surgical scope including illuminating the rear view.

Referring now to FIGS. 2D to 2H, a schematic representation of further example rear-viewable port closure device 300 is shown that generally includes the same aspects and features as rear-viewable port closure devices 100 and 200 except as described herein, which is depicted during usage for an example surgical procedure pertaining to placing one or more sutures within a defect for closing the defect. As such, like numbers refer to like features. The rear-viewable port closure device 300 is shown without exterior cover portions of the cannular housing 210 shown for exposing an arrangement of interior components. Further, the rear-viewable port closure device 300 is depicted with a surgical scope 350 inserted through the cannular housing 250.

The rear-viewable port closure device 300 primarily differs from rear-viewable port closure device 200 in that the obscured light source 390 includes multiple illumination portions 392 including, for example, LEDs located in the tip region 344 of the cannular housing distal portion 334 at lower portions of the cone assembly 345. The plurality of illumination portions 392 can enhance viewability of interior cavity and the reflected rear view during use for the surgical procedure without the illumination portions being directly viewable by the surgical scope. As best seen in FIG. 2G, the illumination portions 392 can be powered by flexible connections routed through the support arms 342 and/or via flexible connections to the surgical scope 350.

As shown in FIG. 2D, at a beginning of the surgical procedure the suture placement assembly 346 can be deployed by rotating the T-Bar about ninety degrees from the longitudinal axis 336. Thereafter, as can be seen in FIG. 2E, the rear-viewable suture placement device 300 can be moved in a proximal direction for placing the deployed suture placement assembly 346 adjacent to the fascial layer for placement of a suture. Optionally, anesthetic can be injected through extendable needles into pre-peritoneum nerves.

FIG. 2F shows the rear-viewable port closure device 300 in a suture placement arrangement with suture material routed through an internal channel including through the suture placement assembly across the defect in a transverse suture pathway region 348 (see FIG. 2G), which is included in the rear view as depicted in FIG. 2H. The rear view 358 can fully include the reflector 366 and the reflected image shown thereon as received by the lens end 352 of the surgical scope 350, as well as portions of the interior cavity 330 falling within the breadth of the lens view. The rear view / reflected image can have a magnification factor 362 corresponding with a degree for which the reflected image is magnified or diminished depending on factors such as a type and shape of the reflector (e.g., concave, convex and/or flat) and a distal offset distance of the reflector 358 from a distal end of the suture placement assembly 346, which can be fixed distance for an arrangement with the reflector disposed in a fixed position and/or an adjustable distance as discussed further below.

As discussed further below, a concave shape for the reflector can be operable for magnifying the reflected rear view image and, thus, can have a magnification factor greater than 1.0, such as 1.5 or 2.0 for magnifying the reflection 150% or 200%. The rear view can further be magnified by the surgical scope for enhancing viewability for the user. A convex shape for the reflector can be operable for diminishing the reflected rear view image along with increasing the span 360 for the rear view so as to extend beyond a width of the suture placement assembly 346. As such, the magnification factor 362 can be less than 1.0, such as 0.5 for reducing the reflection by 50%. However, even though diminished and having an increased span, the rear view with increased span can be magnified for viewability by the surgical scope. As also shown in FIG. 2H, a breadth of the scope view received by the lens end 352 can be wider than a diameter or length and width of the reflector, such that the scope view can show distal portions of the interior cavity beyond perimeter regions of the reflector.

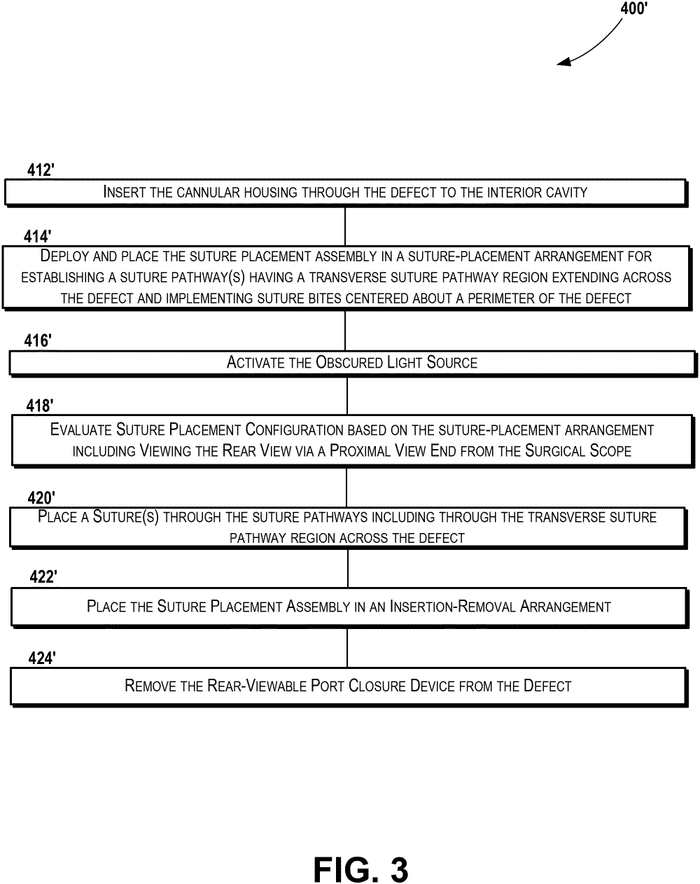

Referring now to FIG. 3, a Method 400′ is schematically represented for performing rear-viewable, in vivo, suture placement and port closure using a rear-viewable port closure device, such as rear-viewable port closure devices 100, 200 and/or 300 described above or further examples described below. Method 400′ can include the actions of 412° inserting the cannular housing through the defect to the interior cavity, 414° deploying and placing the suture placement assembly in a suture-placement arrangement for establishing one or more suture pathways each having a transverse suture pathway region extending across the defect and implementing suture bites centered about a perimeter of the defect, and 416° activating the obscured light source.

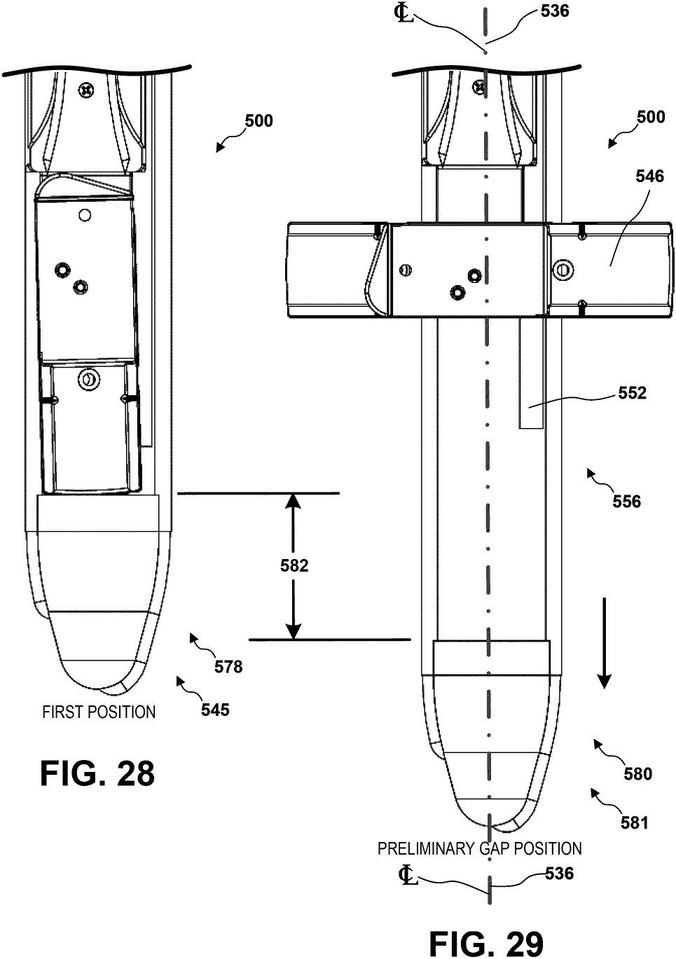

With the obscured light source activate, the method can further include the action of 418 evaluating a suture placement configuration based on the suture-placement arrangement including viewing the rear view via a proximal view end from the surgical scope, which can include adjusting the rear view as discussed further below along with FIGS. 28 to 36. The method can continue with the action of 418° placing a suture through each of the one or more suture pathways including through the transverse suture pathway region across the defect, 420° placing the suture placement assembly in an insertion-removal arrangement, and 422° removing the rear-viewable port closure device from the defect.

Referring now to FIGS. 4A and 4B, a schematic representation of an additional example rear-viewable port closure device 400 is shown that generally includes the same aspects and features as rear-viewable port closure devices 100, 200 and 300 except as described herein, which is depicted as a longitudinal cross-sectional view through the example rear-viewable port closure device. As such, like numbers refer to like features. The rear-viewable port closure device 400 is depicted in FIG. 4A without a surgical scope inserted and in FIG. 4B with a surgical scope 450 inserted through the cannular housing 410, and without showing an obscured light source, which can include an obscured light source, for instance, as described above for device 100, 200 and/or 300. The rear-viewable port closure device 400 primarily differs from rear-viewable port closure devices 200, 300 and 400 in that the reflector 466 is arranged for longitudinal translation and adjustment by the user, and the scope channel 438 is defined as a central scope channel through the cannular housing 310.

The rear-viewable port closure device 400 can include a plurality of reflector support arms 442 arranged to extend parallel with the longitudinal axis of the cannular housing, and the cannular housing 410 can define a corresponding plurality of support arm channels 440 therein. As shown in FIG. 4B, the plurality of reflector support arms 442 can translate within the support arm channels 440 for enabling slidable adjustment of the reflector. The slidable adjustment can include, for instance, a plurality of pre-established positions such as a set position for enabling deployment of the suture placement assembly and positions for optimal viewability of the rear view, as well as an adjustable range for user adjustment of the reflector.

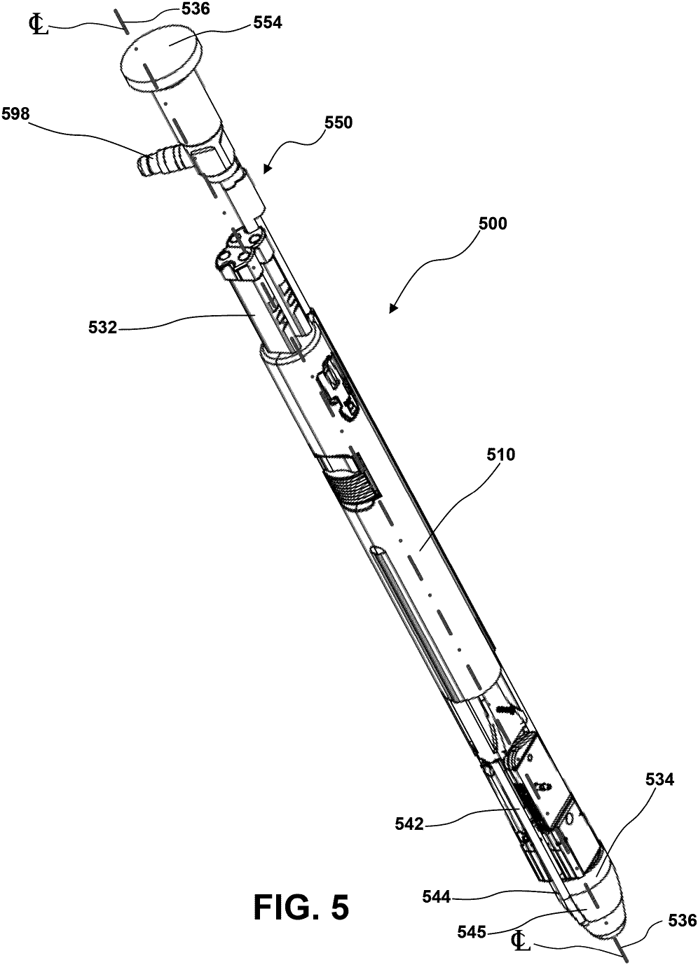

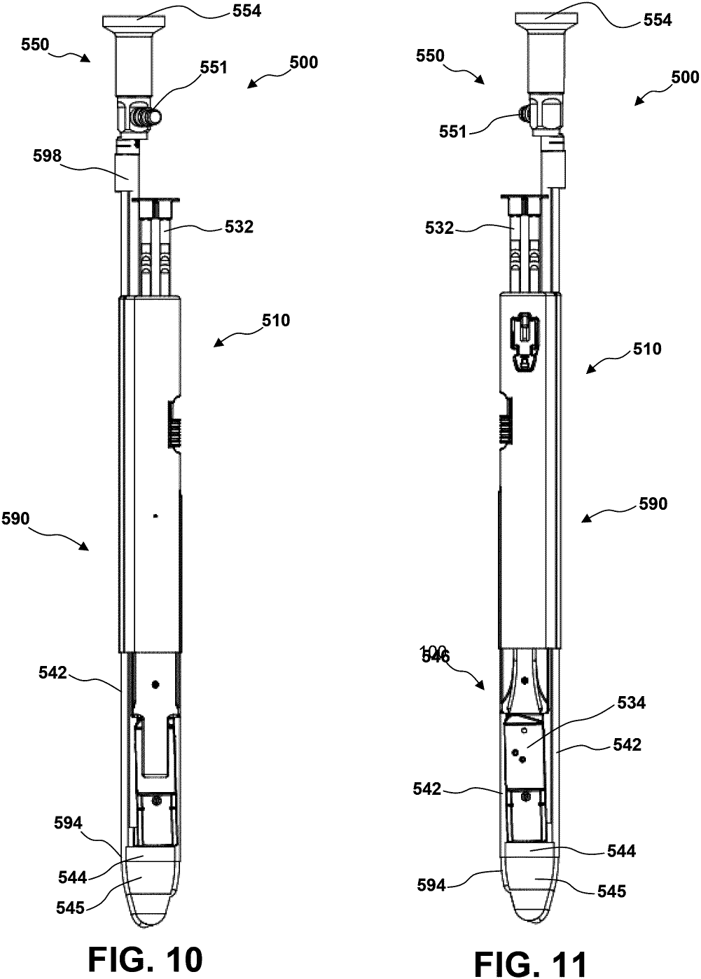

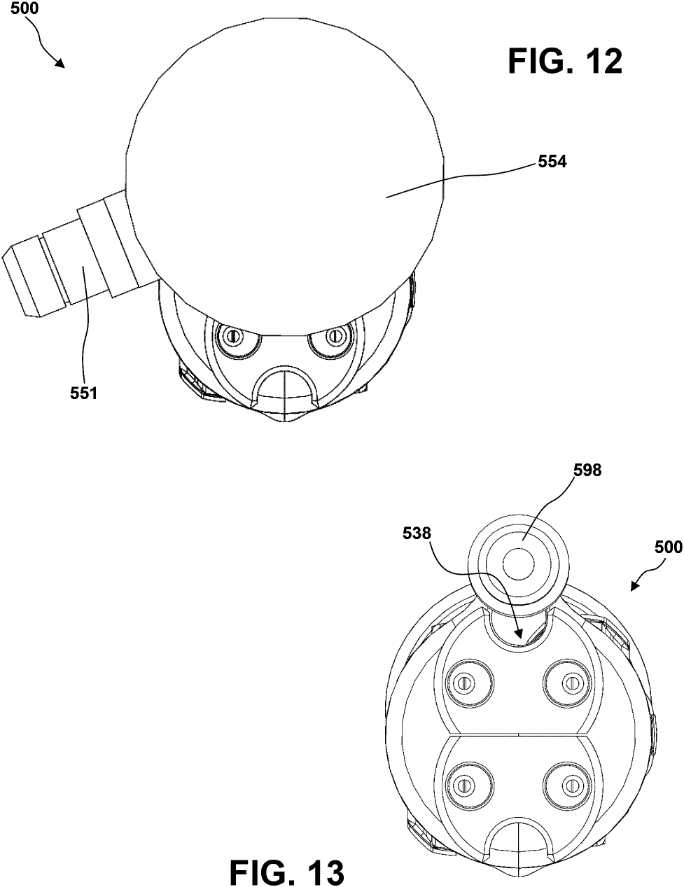

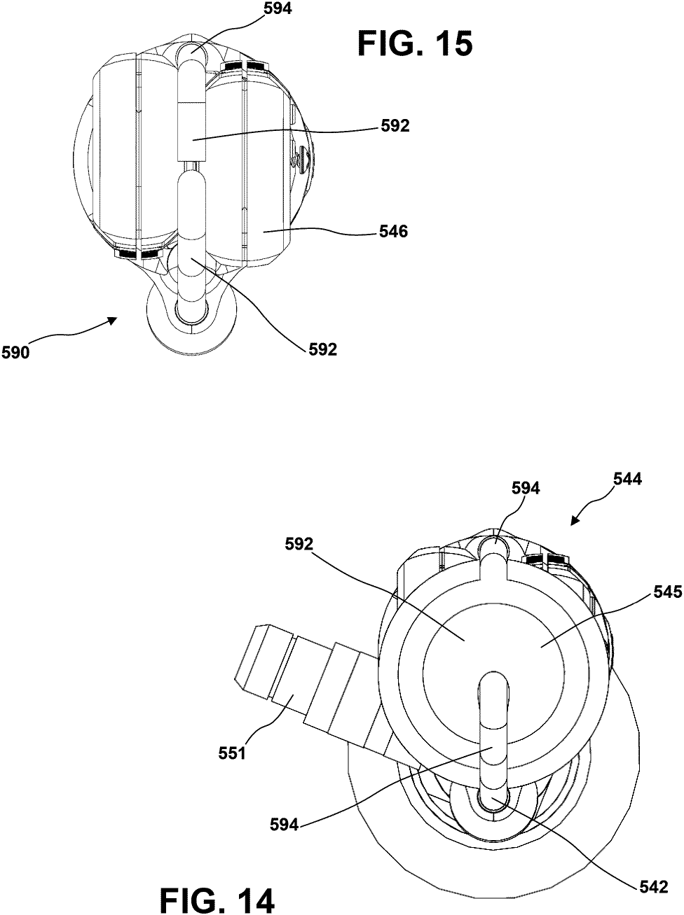

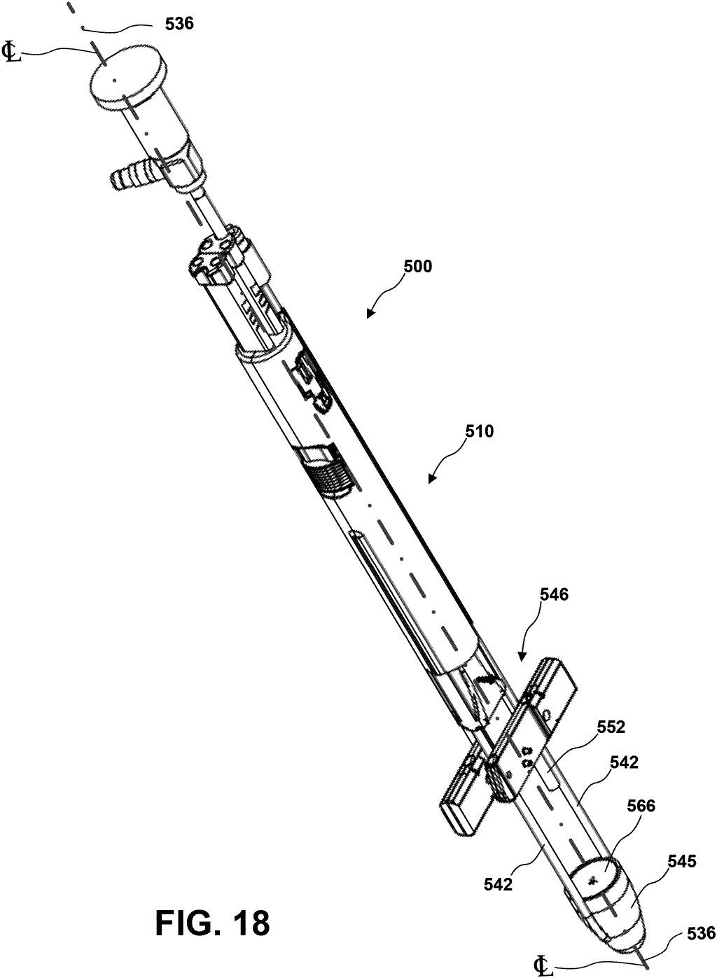

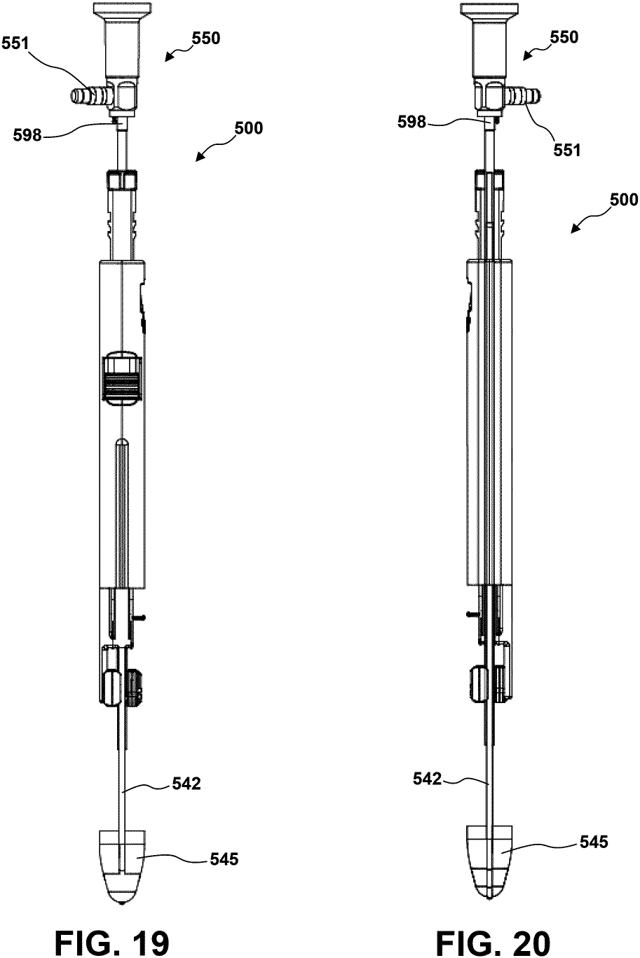

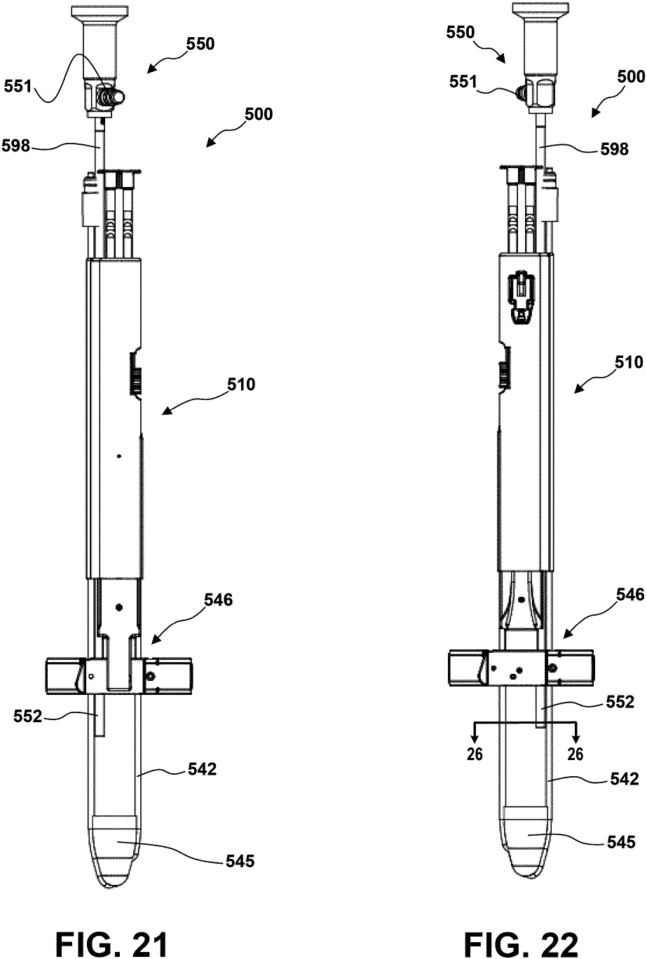

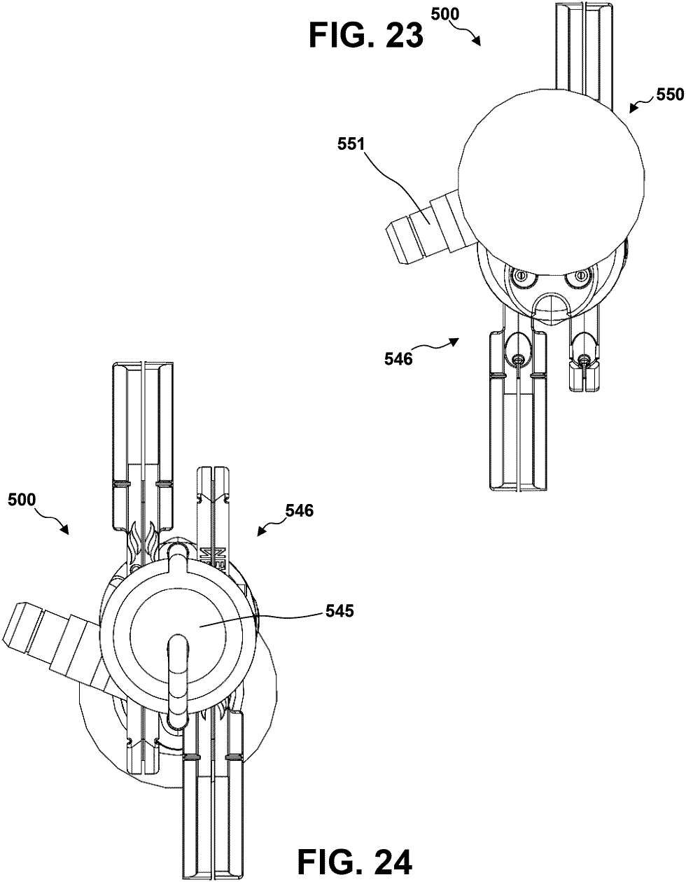

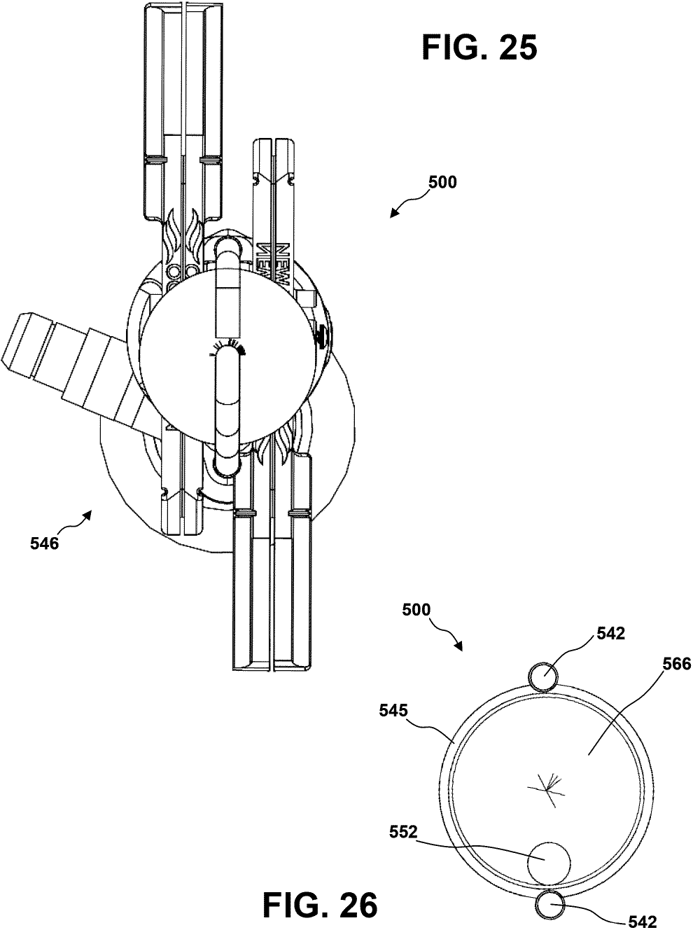

Referring now to FIGS. 5 to 35, a schematic representation of another example rear-viewable port closure device 500 is shown that generally includes the same aspects and features as rear-viewable port closure devices 100, 200, 300 and 400 except as described herein. As such, like numbers refer to like features. The rear-viewable port closure device 500 is shown without a front, distal cover portion of the cannular housing 510 shown for exposing portions of the suture placement assembly 546. Further, the rear-viewable port closure device 500 is depicted with a surgical scope 550 inserted through the cannular housing 550. The rear-viewable port closure device 500 primarily differs from rear-viewable port closure devices 100, 200, 300 and 400 in that the obscured light source 590 includes an arrangement including a light pipe and partially translucent dome assembly for indirectly, glowing illumination of the interior cavity. Like the devices described above, the rear-viewable port closure device 500 can include a cannular housing 510, a suture placement assembly 546, a scope channel 538 defined through the cannular housing, a reflector 566, and an obscured light source 590 connected to the cannular housing.

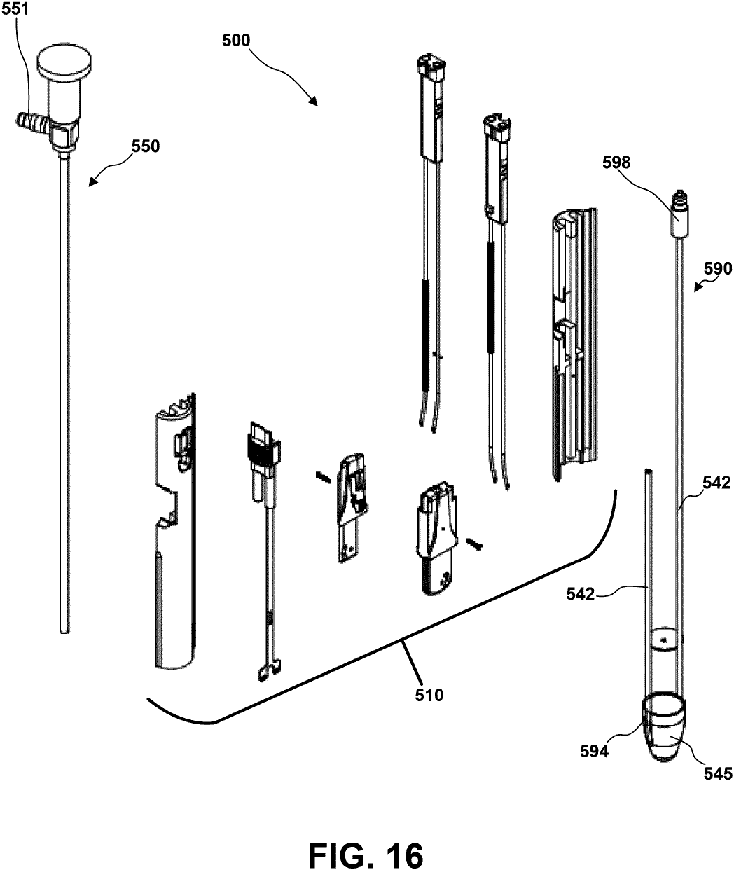

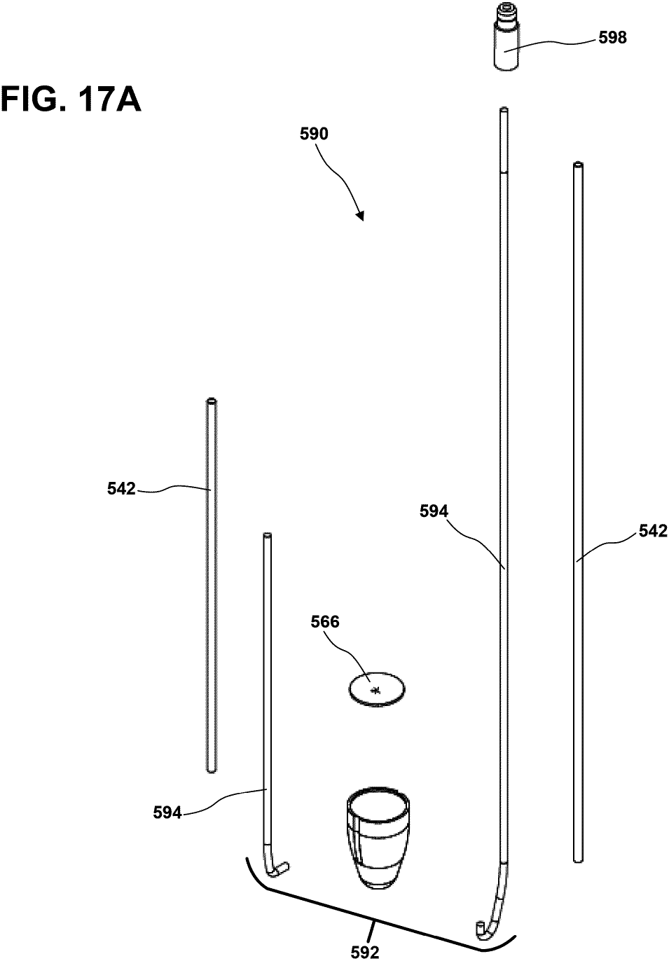

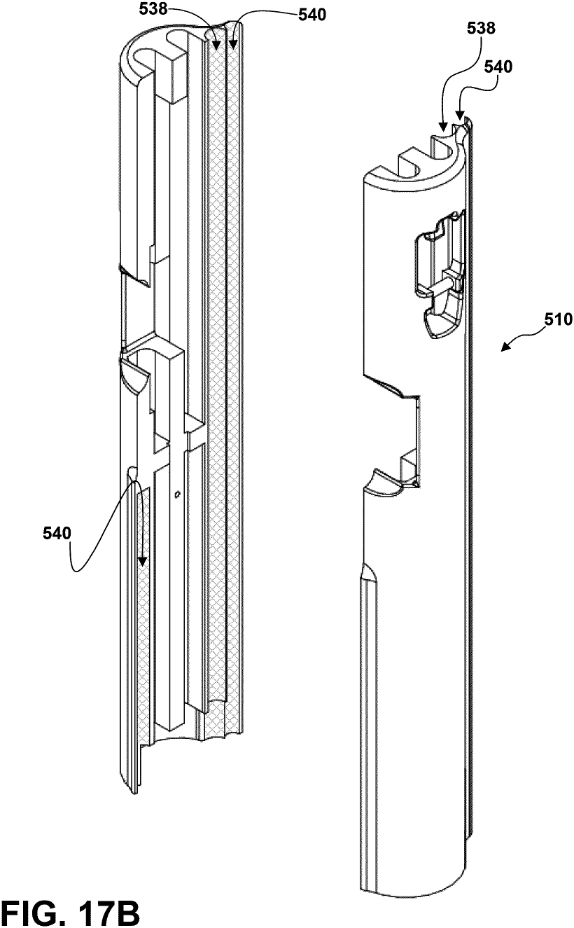

The cannular housing 510 can generally extend a length of the rear-viewable port closure device 500 and include a proximal portion 532, an opposite distal portion 534, and a longitudinal axis 536 oriented therebetween. As shown in FIG. 16, the cannular housing 510 can include opposing exterior panels mated together to form a shell for the cannular housing, the suture placement assembly 546, internal components for enabling device operations including for deploying and returning the suture placement assembly, and portions of a dome assembly 545 that support the reflector 566 and are connected to the shell of the cannular housing. As best seen in FIG. 17B, the scope channel 538 can be defined through the cannular housing 510 including through the shell, which is operable for receiving a lens end 552 of a surgical scope 550 and retaining the lens end at a view position 556 along the longitudinal axis 536 of the cannular housing. As further shown in FIG. 17B, one or more support arm channels 540 can also be defined through the cannular housing including through the shell. The support arm channels can include a support arm channel defined from the proximal portion 532 to the distal portion 534 of the cannular housing, a support arm channel defined from a middle portion of the cannular housing to the distal portion 534, and/or a combination of support arm channels including one of each as is depicted in FIG. 17B. A combination of support arm channels as depicted in FIG. 17B can enable the use of an extended reflector support arm arranged for connecting to a power or light source at a proximal end thereof along with one or more shorter reflector support arms arranged for providing stable support for the reflector during use including for translation with respect to the cannular housing.

The suture placement assembly 546 can be connected to the cannular housing 510 at the distal portion 534, and the reflector 566 can be located at a tip region 544 of the distal portion. The reflector 566 can have a proximal reflective surface 568 and an opposite distal surface 570, in which the proximal reflective surface can be oriented substantially perpendicular to the longitudinal axis. The proximal reflective surface 568 can be operable to reflect a rear view of the suture placement assembly 546 to the lens end 552. The obscured light source 590 can be connected to the cannular housing 510 and can include an illumination portion 592 disposed outside of the rear view and outside of a viewable span of the lens end 552 as discussed above along with FIG. 2H. The illumination portion 592 can be operable for illuminating the rear view and the viewable span when the illumination portion is activated for providing illumination within the interior cavity, such that, when the illumination portion is activated, the rear view and the viewable span are illuminated and viewable by the surgical scope 550 without the illumination portion viewable through the surgical scope.

The suture placement assembly 546 can include one or more rotatable T-bars that can be arranged in a compact insertion-removal arrangement for insertion through the defect and removal from the defect, for which rear-viewable port closure device 500 includes a pair of T-bars. Each T-bar can be rotated about ninety degrees in a deployment arrangement for forming, along with corresponding elongate channels defined through the cannular housing 510, a continuous suture placement channel through the device for placement of a corresponding suture. Each suture placement channel can extend from an input opening at the distal portion 534 through the cannular housing 510 to a first lateral region of a corresponding T-bar, across the defect through the transverse suture pathway region 548 to an opposite lateral region of the corresponding T-bar, and to an output opening at the proximal portion of the cannular housing.

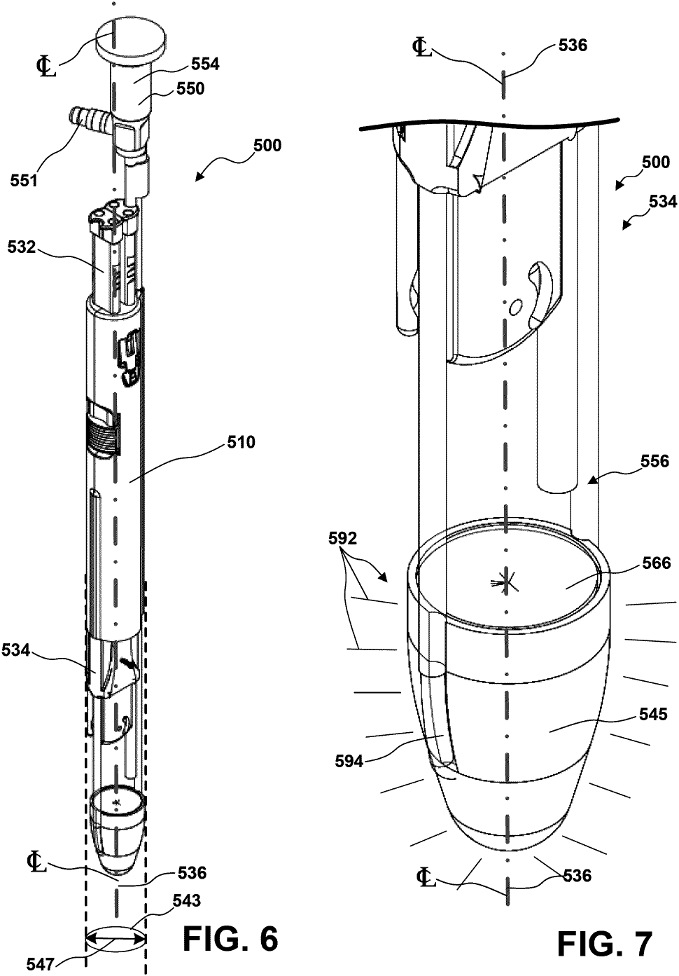

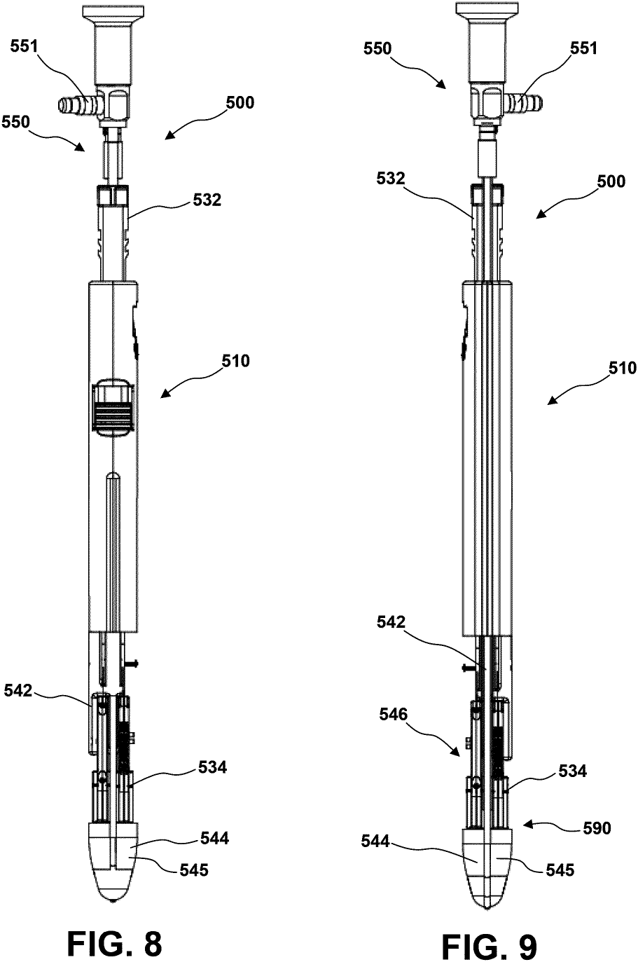

As best seen in FIG. 6, the suture placement assembly 546 can be disposed in the compact insertion-removal arrangement such that the one or more T-bars are oriented parallel with the longitudinal axis 536 of the cannular housing 510. A perimeter 547 of the suture placement assembly while in the insertion-removal arrangement shown in FIG. 6 can be substantially the same and/or fit within an elongate housing envelope 543 defined by the cannular housing 510. In other words, the suture placement assembly 546 can fit within an overall cross-sectional footprint or envelope of the cannular housing for ready insertion through a defect, such as an incision created for single port laparoscopic procedures. As shown in FIGS. 8 to 12, the reflector 566 and dome or cone assembly 545 can be nested closely with and adjacent to a distal end of the suture placement assembly 546 while in the insertion-removal arrangement, which can avoid tissue snags during insertion and removal actions of the device into and out of the defect.

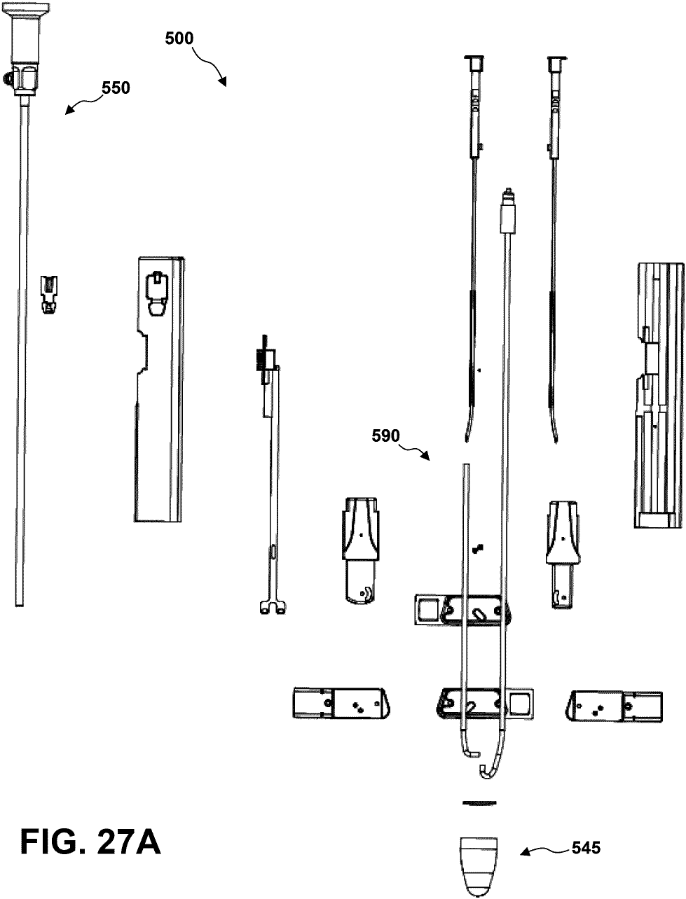

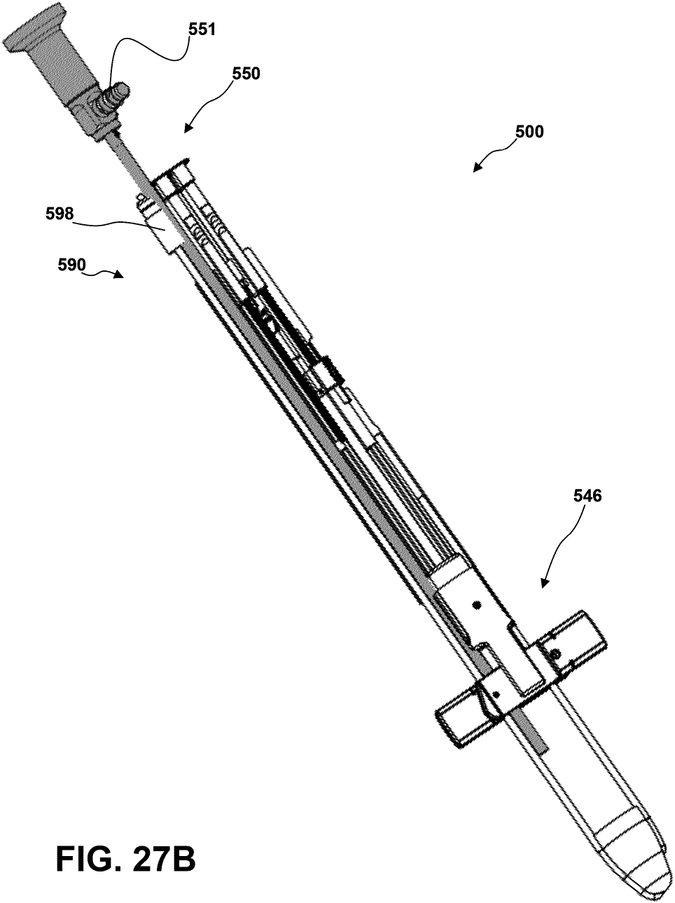

As best seen in FIG. 17B along with FIG. 27B, the scope channel 538 defined through the cannular housing can be arranged for receiving the lens end 552 and the body of the surgical scope 550 through the scope channel. The lens end 552 can be retained at a view position 556 through the cannular housing 510, such that the rear view 558 can be received at the proximal view end 554. The view position 556 can be set location or can be an adjustable position, such that the user can extend and retract the lens end 552 of the surgical scope 550 as desired. The surgical scope 550 can include an adaptor or video connector 551. which can be arranged for connection to video display, recording device, and/or power supply during use of the surgical scope. operating the surgical scope.

As best seen in FIGS. 6 to 9 as an optional arrangement, the view end 554 of the surgical scope 550 proximate the video connector or adaptor 551 can be arranged for closely nesting with and connecting to an adaptor 598 located at a proximal end region of the obscured light source 590 for providing a light source or power connection for operating the obscured light source 590. Further, as depicted in FIG. 27B, the adaptor 598 for the obscured light source 590 can be spaced apart from the surgical scope 550 or otherwise exposed while the device is in a deployed position, such that the adaptor 598 can be connected to a light source or power supply (not shown) for activating and powering the obscured light source 590 independent from the surgical scope 550.

As best seen in FIG. 17A, the obscured light source 590 can be arranged as one or more partially covered light pipes 594, which can include first light pipe 594 and a second light pipe 595. The first light pipe 594 can be connected to the adaptor 598 at a distal end region thereof and extend through a reflector support arm channel 540 defined through the cannular housing 510. The first light pipe 594 can connect at a distal end region to a side region of the dome or cone assembly 545 located at the tip region 544. Similarly, the second light pipe 595 can connect to an opposite or different side region of the dome or cone assembly 545 at distal end region of the second light pipe. The second light pipe 595 can extend upward or distally from the connection with the dome or cone assembly 545 and extend through a second support arm channel defined through at least a portion of the cannular housing 510. The dome or cone assembly 545 can formed from a similar material as each of the first light pipe 594 and the second light pipe 595. As such, the first light pipe 594 can transmit light provided at the adaptor 598 through the first light pipe and to each of the second light pipe 595 and the cone assembly 545 therebetween, such that each of the light pipes and the cone assembly can glow when the obscured light source 590 is activated for illuminating the interior cavity.

With continued reference to FIG. 17A, an assembly for the one or more reflector support arms 542 with the first and second light pipes 594,595 can form the illumination portions 592 of the obscured light source 590 along with supporting the reflector in a translatable or adjustable reflector arrangement. Accordingly, a first reflector support arm 542 can be arranged as a hollow tube or sleeve 542 mated with the first light pipe 594. The first light pipe 594 can extend through the first reflector support arm 542, which can substantially cover an extent of the light pipe from the proximal region adjacent to the adaptor 598 to a connection of the first light pipe 594 to the dome or cone assembly 545. Likewise, a second reflector support arm 542 can be arranged as a hollow tube or sleeve 542 mated with the second light pipe 595, such that the support arm can cover an extent of the second light pipe connected to the cone assembly 545 to a distal end region thereof.

As such, the illumination portion 592 can be provided as a plurality of illumination portions or rather an overall illumination portion 592 formed as exposed portions of the first and second light pipes 594,595 and the dome assembly 545, which can provide a substantially uniform glow region about the cone assembly 545 as the illumination portion 592. The reflector 566 is attached to the cone assembly 545 at a proximal end of the cone assembly, such that the illumination portion 592 arranged as the glow region is disposed at a distal side of the reflector, which can block direct light from the glow region to the lens end 552 while illuminating the interior cavity.

Referring now to FIGS. 28 to 35, translation adjustments and positions for the reflector 566 and cone assembly 545 are generally shown, which can be arranged as pre-set positions and/or adjustable positions determined by the user. FIGS. 28 and 29 depict, as a comparison, translation of the cone assembly 545 to a second position 580 (e.g., a preliminary gap position 581 shown in FIG. 29) from a first position 581 (FIG. 28) corresponding with the compact insertion-removal arrangement, in which the reflector 566 and a proximal side of cone assembly 545 are nested closely with and located adjacent to the suture placement assembly 546, such as prior to deployment of the suture placement assembly. As shown in FIG. 29, the cone assembly 545 and reflector 566 can be translated distally away from the first position by the user to the preliminary gap position 581 by a gap distance 582. The preliminary gap position can be a pre-set or location indicated to the user that is spaced apart from the suture placement assembly 546 a sufficient distance (e.g., gap distance 582) for readily enabling movement of the suture placement assembly 546 including rotation of the T-bars ninety degrees to the deployment position for placing sutures.

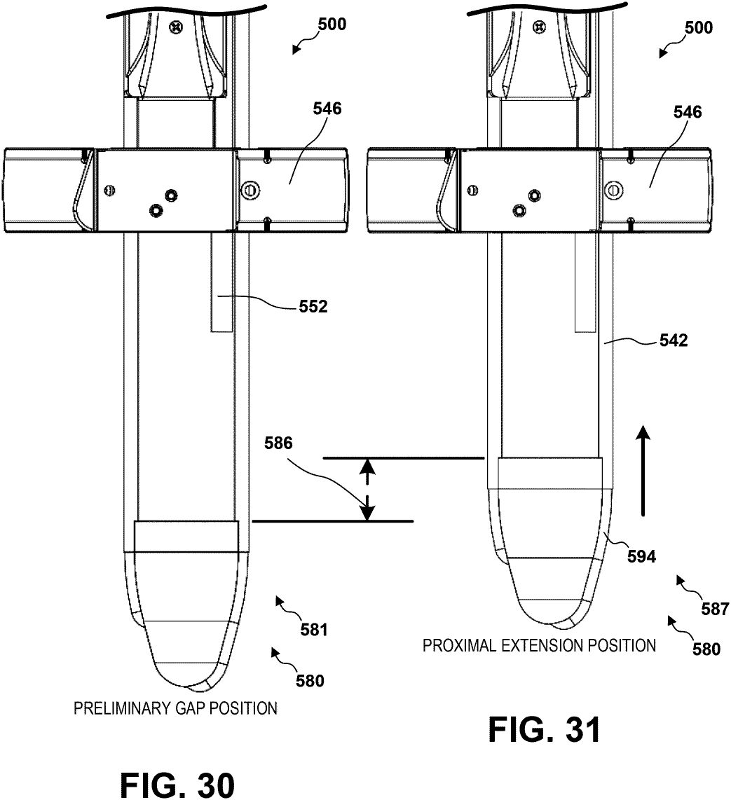

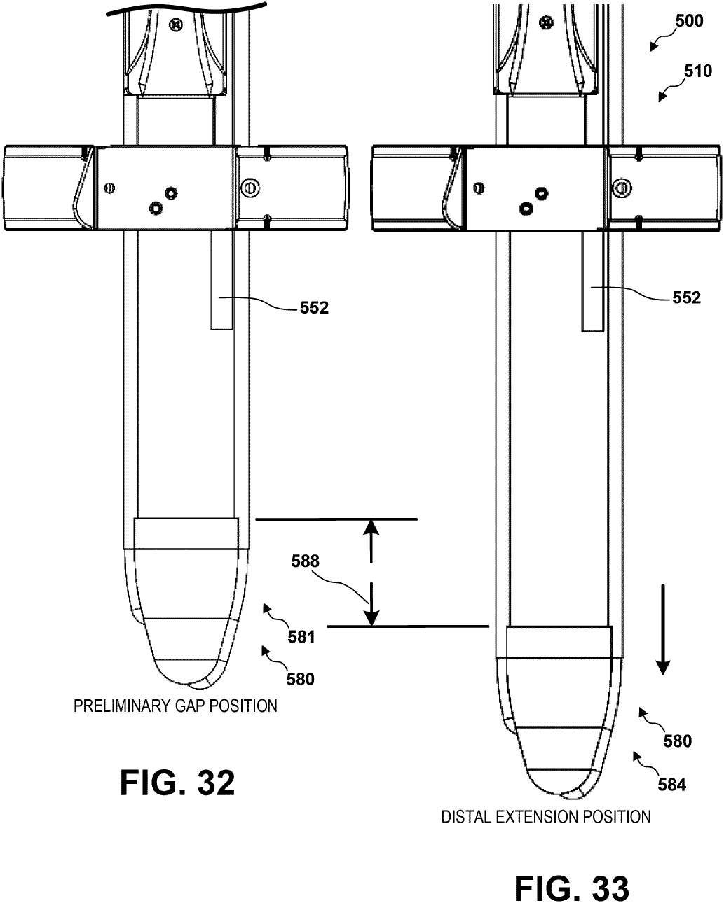

FIGS. 30 and 31 similarly depict, as a comparison, translation of the cone assembly 545 and the reflector 566 from the preliminary gap position 581 to another adjustment or second position 580, such as a proximal extension position 587, which is located proximally from the preliminary gap position by a first distance 586. Movement to the proximal extension position can be made by the user for adjusting the reflector 566 in the proximal direction, such as for fine-tuning the rear view provided to the lens end 552. Likewise, FIGS. 32 and 33 depict, as a comparison, translation of the cone assembly 545 and the reflector 566 from the preliminary gap position 581 to yet another adjustment or second position 580, such as a distal extension position 584, which is located distally away from the preliminary gap position by a second distance 588. Movement to the distal extension position can be made by the user for adjusting the reflector 566 in the distal direction, such as for fine-tuning the rear view provided to the lens end 552.

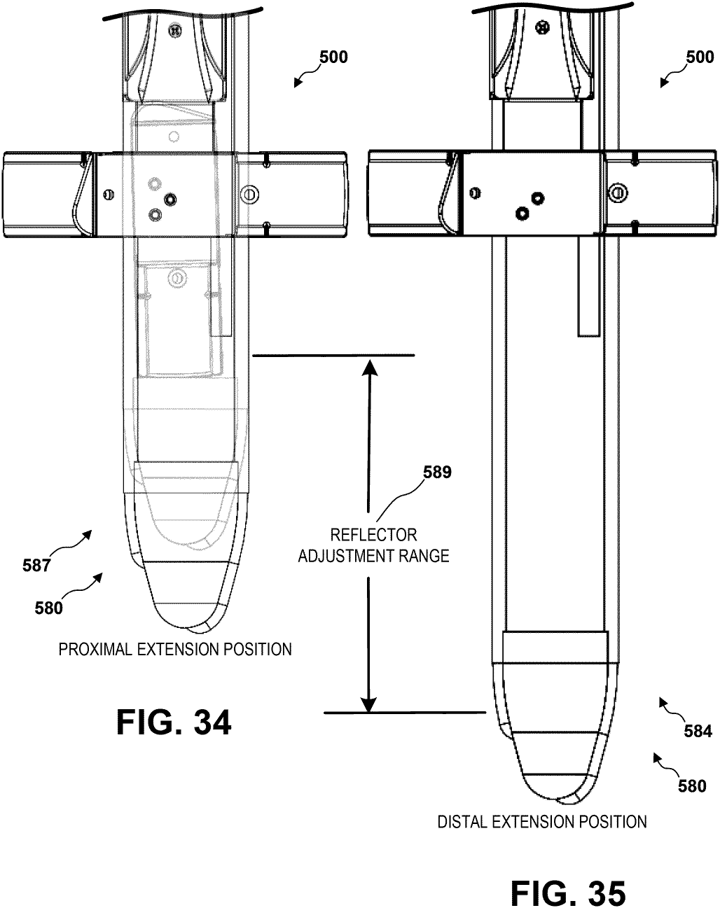

It is understood that the user is not required to make such movements and adjustments except as may be needed for enabling movement of the suture placement assembly 546 to the deployed position, which may not require movement to or use of the preliminary gap position 581. For instance, the user can choose to translate the reflector 566 and cone assembly 545 from the first position 578 directly to one of the proximal extension position 587 or the distal extension position 584 and/or to another translation position within a range of movement for the reflector and the cone assembly. Further, it is understood that a distance between the closest identified position (proximal extension position 587) and the furthest identified position (distal extension position 584) can fall within a reflector adjustment range 589 shown by way of comparison in FIGS. 34 and 35, but that the reflector adjustment range can extend beyond the identified distal extension position 584 and also closer than the first position 578 after the suture placement assembly 546 has been rotated and deployed.

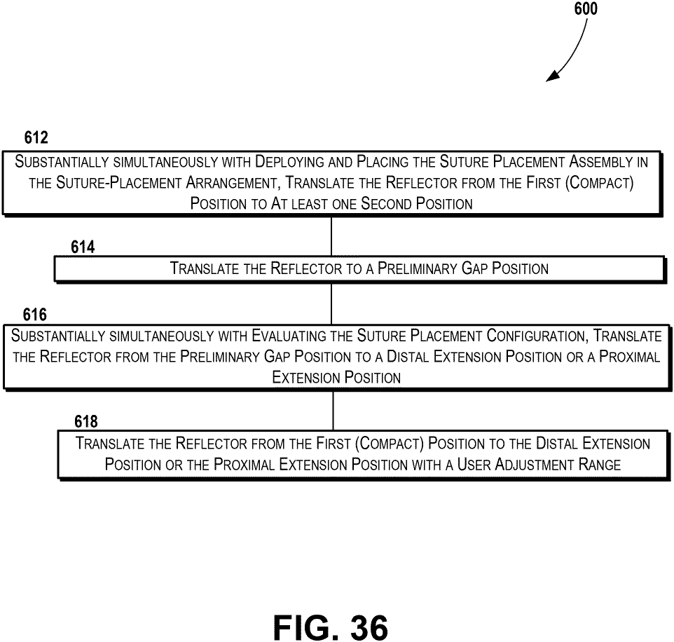

Referring now to FIG. 36, a Method 600 is schematically represented for adjusting the reflector position and corresponding rear view provided to the lens end of the surgical scope, which can be included as part of Method 400′ described above. As such, Method 600 can include the actions of 612 substantially simultaneously with deploying and placing the suture placement assembly in the suture-placement arrangement, translating the reflector from the first (compact) position to at least one second position, and 614 translating the reflector to a preliminary gap position. Method 600 can further include the action of 616 substantially simultaneously with evaluating the suture placement configuration, translating the reflector from the preliminary gap position to a distal extension position or a proximal extension position.

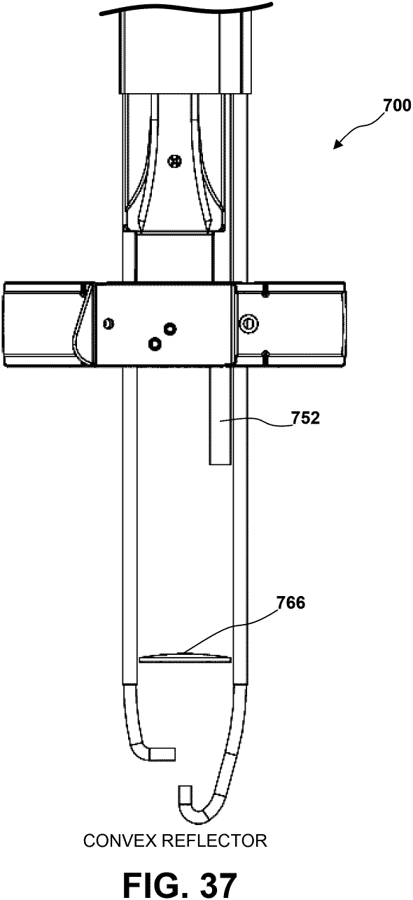

Referring now to FIG. 37, a schematic representation of an additional example rear-viewable port closure device 700 is shown that generally includes the same aspects and features as rear-viewable port closure devices 500 except as described herein. As such, like numbers refer to like features. Only distal portions of rear-viewable port closure device are shown in FIG. 37, for which the cone portion of the cone assembly 745 has been omitted for exposing the reflector 766. The rear-viewable port closure device 700 primarily differs from the rear-viewable port closure device 500 in that the reflector is arranged as a convex reflector 766, such that the proximal reflective surface oriented toward the lens end 752 has a convex shape. As discussed above, the use of a convex shape for the reflector can provide advantages including reflecting a diminished rear view to the lens end that has a magnification factor of less than 1.0, such as about 0.5. As such, similar to operation of vehicle rear view mirror, an increased span can be provided for the rear view that can enable the user to view an increased distal region about the suture placement device, such increased perimeter regions about the defect. The diminished rear view can nonetheless be magnified for enabling sufficient detail view for the user, such as via magnification that can be provided by the surgical scope.

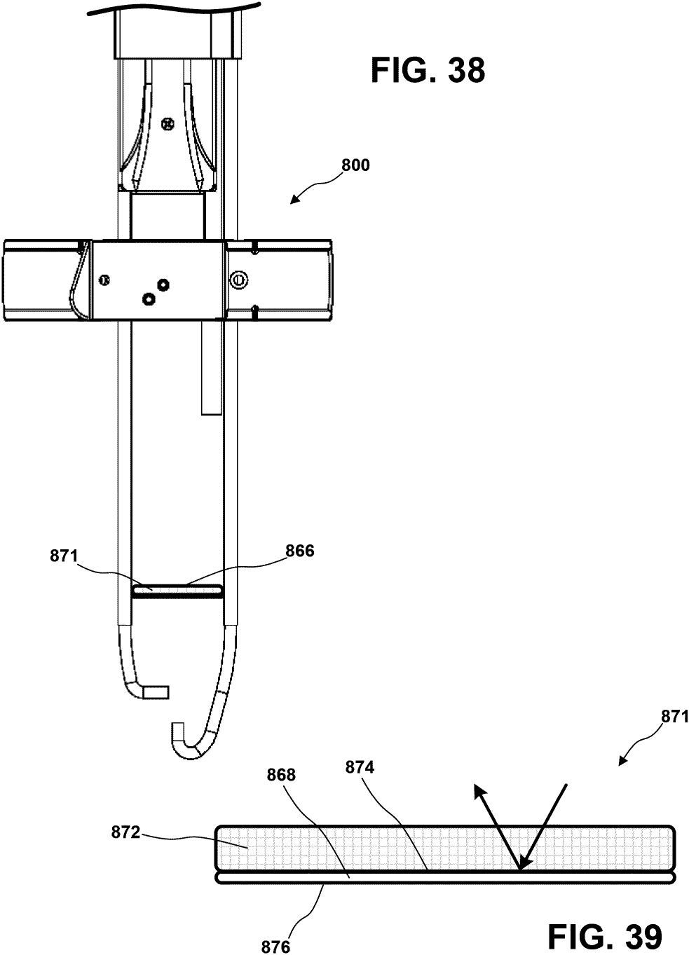

Referring now to FIGS. 38 and 39, a schematic representation of yet another example rear-viewable port closure device 800 is shown that generally includes the same aspects and features as rear-viewable port closure devices 500 and 600 except as described herein. As such, like numbers refer to like features. Similar to FIG. 37, FIG. 38 only shows distal portions of rear-viewable port closure device 800, for which the cone portion of the cone assembly 845 has likewise been omitted for exposing the reflector 866. The rear-viewable port closure device 600 primarily differs from the rear-viewable port closure devices 500 and 600 in that the reflector is arranged as a flat reflector 866, such that the proximal reflective surface oriented toward the lens end 852 is a real image having a magnification factor of about 1.0.

Further, as best seen in FIG. 39, the flat reflector 866 is depicted as a mirror 866. As noted above, the reflector described for any of the above rear-viewable port closure devices and according to aspects and features described herein can make use of various shapes, sizes and arrangements of reflectors, which can include the use of traditional type mirror 871. As shown in FIG. 39, when the reflector is arranged as mirror 871, the reflector 866 can include a transparent sheet 872, such as a sheet formed from glass, plexiglass, LEXAN, a polymeric sheet or other substantially transparent sheet. A rear or distal material or coating 868 on a distal surface of the transparent sheet 872 can form the proximal reflective surface 874 oriented toward the lens end 852, and the distal surface 876 can be substantially opaque and/or partially transparent or translucent, such as is used for one-way mirrors and the like.

The subject matter described above is provided by way of illustration only and should not be construed as limiting. Various modifications and changes can be made to the subject matter shown or described herein without following the example configurations, arrangements and applications illustrated or described, and without departing from the true spirit and scope of the inventive aspects, features, concepts and technologies disclosed herein.

Parts List

| | | |
|---|---|---|
| No. | Part | Comment; Potential options |
| x00 | Rear-viewable Port Closure Device | |
| x02 | Suture material | |
| x10 | Cannular Housing | Includes suture assy. support/covers |
| x20 | Patient’s skin | |
| x22 | Fat layer | |
| x24 | Fascia layer | |
| x26 | Inert gas | |
| x28 | Defect | Surgical port; Incision; Wound |
| x30 | Interior Cavity | Created/Expanded via inert gas |
| x32 | Proximal portion (housing) | Physician/User end / User control end |
| x34 | Distal Portion (housing) | E.g., in vivo region |
| x36 | Longitudinal Axis (housing) | |
| x38 | Scope Channel | |
| x40 | Support arm channels | |
| x42 | Reflector support arms | |
| x43 | Elongate housing envelope | Housing perimeter |
| x44 | Tip region of housing distal portion | Lead end; Blunt tip |
| x45 | Cone assembly | Blunt tip |
| x46 | Suture placement Assembly | |
| x47 | Perimeter (of suture placement assy) | (Insertion-removal arrangement) |
| x48 | Transverse suture pathway region | Portion across port/defect/wound |
| x50 | Surgical/Suture Scope | E.g., endoscope; laparoscope |
| X51 | Scope connector | E.g., display/power connector |
| x52 | Lens end (scope) | Lens/aperture for capturing images |
| x54 | View end (Scope) | Physician/user end of scope |
| x56 | View position (lens end) | Preset or adjustable location in channel |
| x58 | Rear view | View reflected to scope from mirror |
| x60 | Rear view span | E.g., circular diameter or image I/w |
| x62 | Magnification factor | Concave >1.0; Convex <1.0; Flat 1.0 |
| x64 | Lens view breadth | E.g., circular diameter or view I/w |
| x66 | Reflector | E.g., mirror; reflective metallic sheet |
| x68 | Proximal reflective surface | (Facing scope/user) |
| x70 | Distal surface (reflector) | Distal surface (reflector) |
| x71 | Mirror | Transp. sheet w/ reflective/opaque surf. |
| x72 | Transparent sheet (mirror) | E.g., glass; plexiglass; polymer sheet |
| x74 | Transparent prox. surface (mirror) | Mirror construction |
| x76 | Distal opaque surface (mirror) | E.g., opaque, translucent, two-way |
| x78 | First position (reflector) | E.g., compact insertion-removal arr. |
| x80 | Second position (reflector) | E.g., for deployment or user-selectable |
| x81 | Preliminary gap position | E.g., Translated for deployment |
| x82 | Gap distance (prelim. gap position) | E.g., set distance or user-selected |
| x84 | Distal extension position (reflector) | E.g., user-adjustable or set positions |
| x86 | First distance | E.g., distal from prelim. gap position |
| x87 | Proximal extension position | E.g., user-adjustable or set positions |
| x88 | Second distance | E.g., proximal from prelim. gap posn. |
| x89 | Reflector adjustment range | |
| x90 | Obscured Light Source | E.g., LED; Partially covered light pipe |
| x92 | Illumination Portion | |
| x94 | Light pipe | Light pipe |
| X95 | Second light pipe | |
| x96 | Opaque exterior | E.g., opaque coating, outer sleeve |
| x98 | Adaptor (power; light source) | E.g., light source and/or power for LED |

We claim:

1. A rear-viewable port closure device comprising:
a cannular housing operable for percutaneous insertion through a defect to an interior cavity of a body, the cannular housing having a proximal portion, an opposite distal portion, and a longitudinal axis oriented therebetween;
a suture placement assembly connected to the cannular housing at the distal portion;
a scope channel defined through the cannular housing operable for receiving a lens end of a surgical scope and retaining the lens end at a view position along the longitudinal axis;
a reflector at a tip region of the distal portion having a proximal reflective surface and an opposite distal surface, the proximal reflective surface oriented substantially perpendicular to the longitudinal axis, the proximal reflective surface operable to reflect a rear view of the suture placement assembly to the lens end; and
an obscured light source connected to the cannular housing having an illumination portion disposed outside of the rear view and outside of a viewable span of the lens end the illumination portion operable for illuminating the rear view and the viewable span when the illumination portion is activated;
wherein, when the illumination portion is activated, the rear view and the viewable span are illuminated and viewable by the surgical scope without the illumination portion viewable through the surgical scope.

2. The rear-viewable port closure device of claim 1, wherein:
the reflector is movable with respect to the cannular housing between a first position and at least one second position, the at least one second position corresponding deployed state of the suture placement assembly; and
the reflector is operable to reflect the rear view to the lens end of the surgical scope when in the at least one second position.

3. The rear-viewable port closure device of claim 2, wherein:
the reflector is translatable parallel to the longitudinal axis with respect to the cannular housing between the first position and the at least one second position; and
the first position corresponds with an insertion-removal state of the suture placement assembly, when in the insertion-removal state the rear-viewable port closure device is operable for insertion through the defect to the interior cavity and for removal from the interior cavity through the defect.

4. The rear-viewable port closure device of claim 3, wherein:
the plurality of second positions comprise:
a preliminary gap position wherein the reflector is distally translated a gap distance apart from the first position for enabling deployment of the suture assembly;
a distal extension position wherein the reflector is distally translated a first distance from the preliminary gap position for providing the rear view to the lens end of the surgical scope; and
a proximal extension position wherein the reflector is proximally translated a second distance from the preliminary gap position for providing the rear view to the lens end of the surgical scope;
wherein the distal extension position and the proximal extension position fall within a reflector adjustment range for enabling user adjustment of at least one of the rear view and the viewable span.

5. The rear-viewable port closure device of claim 3, wherein;
the reflector comprises a concave reflector;
the proximal reflective surface is concave;
the proximal reflective surface is operable to provide the rear view to the lens end as a magnified view having a magnification factor greater than one (I), and
the magnification factor and a breadth of the rear view are adjustable based on the at least one second position.

6. The rear-viewable port closure device of claim 3, wherein:
the reflector comprises a convex reflector;
the proximal reflective surface is convex;
the proximal reflective surface is operable to provide the rear view to the lens end as a diminished view having a magnification factor less than one (1); and
the magnification factor and a breadth of the rear view are adjustable based on the at least one second position.

7. The rear-viewable port closure device of claim 3, wherein:
the reflector comprises a mirror including a transparent sheet having a transparent proximal surface and a distal opaque surface, a proximal face of the distal opaque surface operable to reflect light and an opposite distal face of the distal opaque surface operable to block light;
the mirror comprises one of a concave mirror, a convex mirror, and a substantially flat mirror, the substantially flat mirror is operable to provide the rear view to the lens end as a real image substantially unmagnified and undiminished; and
a breadth of the rear view is adjustable based on the at least one second position.

8. The rear-viewable port closure device of claim 3, wherein the illumination portion is translatable parallel to the longitudinal axis with respect to the cannular housing along with translation of the reflector.

9. The rear-viewable port closure device of claim 1, wherein the illumination portion is operable for indirectly illuminating the rearview and portions of the interior cavity within the viewable span.

10. The rear-viewable port closure device of claim 1 further comprising a plurality of reflector support arms connecting the reflector to the cannular housing, wherein:
the illumination portion is located at at least one of the following locations;
distally behind the distal surface of the reflector; and
at an outboard side of at least one of the reflector support arms.

11. The rear-viewable port closure device of claim 10, further comprising a plurality of support arm channels defined through the cannular housing oriented parallel with the longitudinal axis operable to receive the plurality of the reflector support arms, wherein:
the obscured light source comprises a light pipe extending through one of the plurality of support arm channels and forming one of the plurality of support arms, a region of the light pipe forming the illumination portion, the light pipe having an opaque exterior substantially covering an outer surface of the light pipe without covering the outer surface at the illumination portion.

12. The rear-viewable port closure device of claim 11, wherein a proximal end of the light pipe comprises an adaptor operable for attaching to an illumination source.

13. The rear-viewable port closure device of claim 1, wherein:
an exterior region of the cannular housing extending along the longitudinal axis defines an elongate housing envelope;
the suture placement assembly is movable between an insertion-removal arrangement and a suture-placement arrangement;
a perimeter of the suture placement assembly is substantially the same as or fits within the elongate housing envelope when in the insertion-removal arrangement; and
portions of the suture placement assembly extend laterally away from the longitudinal axis and outside of the elongate housing envelope when in the suture-placement arrangement corresponding with a deployed state of the rear-viewable port closure device for establishing at least one suture pathway including a transverse suture pathway region across the defect; and
the rear view is operable to include the transverse suture pathway region of the at least one suture pathway.

14. The rear-viewable port closure device of claim 13, wherein:
the rear-viewable port closure device is operable for selective placement between the deployed state and a non-deployed state for insertion and removal of the rear-viewable port closure device through the defect to the interior cavity, the non-deployed state comprising the suture placement assembly in the insertion-removal arrangement and the reflector located at the first position;
wherein, the first position is located immediately adjacent to a distal end of the suture placement assembly.

15. The rear-viewable port closure device of claim 1, wherein:
when the rear-viewable port closure device is disposed through the defect and the suture placement assembly is in a suture-placement arrangement operable for establishing at least one suture pathway each comprising a transverse suture pathway region extending across the defect and for implementing suture bites centered about a perimeter of the defect, the rear view is operable to include the transverse suture pathway region and the suture bites for each of the at least one suture pathway.

16. The rear-viewable port closure device of claim 1, further comprising a cone assembly attached to the reflector and located at a distal side of the reflector, the obscured light source connected to the cone assembly, the cone assembly incorporating a material capable of emanating light when illuminated;
wherein when the obscured light source is activated light travels from the obscured light source to the cone assembly such that the cone assembly emanates light.

17. A method for rear-viewable, in vivo, suture placement and port closure using a rear-viewable port closure device as recited in claim 1, the method comprising:
inserting the cannular housing through the defect to the interior cavity;
deploying and placing the suture placement assembly in a suture-placement arrangement for establishing one or more suture pathways each having a transverse suture pathway region extending across the defect and implementing suture bites centered about a perimeter of the defect;
activating the obscured light source;
evaluating a suture placement configuration based on the suture-placement arrangement including viewing the rear view via a proximal view end from the surgical scope;
placing a suture through each of the one or more suture pathways including through the transverse suture pathway region across the defect;
placing the suture placement assembly in an insertion-removal arrangement; and
removing the rear-viewable port closure device from the defect.

18. The method of claim 17, wherein the reflector is operable for selective translation parallel to the longitudinal axis between a first position located adjacent to a distal end of the suture placement assembly when the suture placement assembly is in the insertion-removal arrangement and at least one second position distally translated away from the first position, the method further comprising:
substantially simultaneously with the deploying and placing the suture placement assembly in the suture-placement arrangement, translating the reflector from the first position to the at least one second position; and
substantially simultaneously with the placing the suture placement assembly in the insertion-removal arrangement, translating the reflector from the at least one second position to the first position.

19. The method of claim 18, wherein the at least one second position comprises a plurality of second positions including a preliminary gap position and at least one of a distal extension position and a proximal extension position, the method further comprising:
substantially simultaneously with the deploying and placing the suture placement assembly in the suture-placement arrangement, translating the reflector from the first position to a preliminary gap position wherein the reflector is distally translated a gap distance apart from the first position for enabling deployment of the suture assembly; and
substantially simultaneously with the evaluating the suture placement configuration, translating the reflector from the preliminary gap position to one of the distal extension position and the proximal extension position; wherein:
translating the reflector from the preliminary gap position to the distal extension position includes distally translating the reflector a first distance from the preliminary gap position to the distal extension position;
translating the reflector from the preliminary gap position to the proximal extension position includes proximally translating the reflector a second distance from the preliminary gap position to the proximal extension position;
the distal extension position and the proximal extension position fall within a reflector adjustment range for enabling user adjustment of at least one of the rear view and the viewable span; and
the translating the reflector from the preliminary gap position to one of the distal extension position and the proximal extension position enables user adjustment of at least one of the rear view and the viewable span.

20. The method of claim 18, wherein:
the illumination portion is operable for selective translation parallel to the longitudinal axis along with the selective translation of the reflector; and
each of the translating the reflector from the first position to the at least one second position and the translating the reflector from the at least one second position to the first position comprises correspondingly translating the illumination portion along with translating the reflector.
